SEQUENCE LISTING (36 sequences)

SEQ ID NO: 1 - DNA, 831 bp, Arabidopsis thaliana
atgatcgccgccggagctaaatcgctattagggctttcgatggcttctccaaaagggattttcgatagcaattcaatgagtaattcaagatctgttgttgttgttcgtgcttgtgtttctatggatggatctcaaactctgagtcataacaaaaatggatctattcctgaggttaaatccattaacggtcacacgggacaaaagcaaggacctttgtctacggtcggaaactcgacaaatataaagtggcatgaatgttctgttgagaaagttgatagacagagattgcttgatcagaaaggatgtgtgatttgggtcaccggtcttagtggttcagggaagagtactttggcttgtgctttgaatcagatgttgtatcaaaaggggaagctttgttatattcttgatggtgataatgttaggcatggcttaaaccgtgatcttagctttaaagctgaggatcgtgcagagaatattcgtagagttggagaggttgctaagctttttgcggatgctggaataatctgcattgcgagtttgatatctccttatagaacagatagggacgcttgtcgaagtttgctccccgagggagattttgttgaggtgttcatggatgtaccgcttagtgtttgcgaggcgagggatccaaagggtctttacaagcttgctcgtgcaggaaagatcaaaggttttaccgggatcgatgacccttacgagccaccattgaactgcgagatttctctaggacgtgaaggaggaacttctcctatcgaaatggcggaaaaggtcgtcggatacttagataacaagggttatcttcaagcataa

SEQ ID NO: 2 - DNA, 882 bp, Arabidopsis thaliana
atggaaggattagctatcagagcatctcgaccgtcggttttctgttctattccaggtctcggcggcgattcccaccgaaaacctccaagtgacggtttcctcaagctgcctgcgtcgtctattccggcggacagccgaaaattagtagcgaattctacttcctttcatccaatctcagccgttaacgtctctgctcaagcttccctcaccgctgattttcccgccctttcagaaactatactgaaagagggaagaaataacggaaaagagaaagcagagaacatcgtgtggcacgagagttcgatatgcagatgcgacagacaacaacttcttcaacaaaagggttgtgtcgtttggatcactggtctcagtggttcagggaaaagcactgttgcttgtgcactaagtaaagcattgtttgaaagaggcaaacttacttacacactcgacggcgacaatgtccgtcacggccttaaccgtgacctcactttcaaagcagagcaccgcaccgaaaacattagaagaattggtgaggtggctaagttgtttgctgacgtcggagtcatttgtatagcaagtttgatttctccgtaccggagagacagagacgcgtgccggtccttgttacctgacggcgatttcgtcgaggtcttcatggacgttcctcttcatgtgtgcgagtcgagagatccaaaggggttgtacaagcttgcacgtgcaggcaaaatcaaaggcttcactggaatcgacgacccttacgaggcaccagtgaattgcgaggtagtgctgaaacacacaggagacgacgagtcgtgttcgccacgtcagatggctgagaacatcatctcttacctgcaaaacaaaggttatcttgagggctaa

SEQ ID NO: 3 - DNA, 627 bp, Arabidopsis thaliana
atgtcgacagtgggaaattcaacgaacatattttggcaagaatcccccattgggaaaactgaaaggcagaagttgttaaatcagaagggttgtgtggtgtggatcacagggctcagtggctcagggaaaagcacgttagcttgctcgcttagtagagagctgaacaaccggggaaagctatcatatattcttgatggtgacaatcttcgtcatggtttgaacaaagatcttggtttcaaggcagaggatagagtggaaaatatacgcagggtcggagaagtagccaaactctttgcggatgctggtttaatctgtattgccagcctcatatccccgtatagaaaagaccgtgacgcctgcagggaaatgatacagaattcatcttttattgaggttttcatgaacatgtctctgcaattgtgtgaagcaagggaccctaaaggcctatacaagcttgcacgtgcaggaaagatcaaaggtttcacaggaatagatgatccgtatgagtctcccttgaactgtgagatagagttgaaagagaaagagggagagtgtccttcccctgtagctatggctgaggaagtgatctcttatctagaagacaaaggtttccttcaaaacgagtaa

SEQ ID NO: 4 - DNA, 933 bp, Arabidopsis thaliana
atggatgttgccgcgatggcgagatgtgtgggaaggtgctacgtttcgccggcgttcggtgagtctgaatcgcaccggttatcggagcggcggtttctgaaattgtctagctccacgaattcggatcccgccggtagtaaaagtttgaagcttcgcgggaaaattcatcggagaatgagctacttccgtccgatcatggcaaaagatgaatccatttcatcgcggtccggtgaaacgaagcaaatcaatggaaagcaaaagaacattgtctggcatgattgtcccgttactaaatccgacaggcaagaattaattaagcaaaagggatgtgtgatttggattactggcttaagtggttcaggtaaaagtagtctggcatgtgctcttagtcgagctttgcacaatcgtggaaagctttcgtatatacttgatggtgacaatgttcgacatggtttaaacagcgatcttagtttcgaagcagatgatcgagctgaaaacattcgaagagttggtgaagtggctaaactgtttgcagattctggtattatctgtattgcaagtttaatatctccttaccggatagaacgagctgcctgccgtgcattattaccacaaggagatttcattgaggtatttatggatgtgccactccatgtttgtgaagctagagatccaaagggcttatacaaacgtgcacgcgctggtaaaatcaaaggttttacaggagtagatgatccatatgaagcgcctttggattgcgagattgtaatacaaaacagtagagacaaggggctttcttcttcatcttcatcttcatcttcaccttcatcttcgtcttcttctctgtgtgaaatggcagatattgttgtgtcgtacttggaccaaaatggatacctgaagaaacactccacaaaatcacgtaattgtatgtaa

SEQ ID NO: 5 - DNA, 23 bp, Artificial Sequence
  Primer
ccacaactgcatatagactatgc

SEQ ID NO: 6 - DNA, 18 bp, Artificial Sequence
  Primer
gacccaaatcacacatcc

SEQ ID NO: 7 - DNA, 18 bp, Artificial Sequence
  Primer
taacgtctctgctcaagc

SEQ ID NO: 8 - DNA, 18 bp, Artificial Sequence
  Primer
atgttttcggtgaggtgc

SEQ ID NO: 9 - DNA, 26 bp, Artificial Sequence
  Primer
gcgcatatgtcgacagtgggaaattc

SEQ ID NO: 10 - DNA, 22 bp, Artificial Sequence
  Primer
ctatcactacatatgagacacc

SEQ ID NO: 11 - DNA, 20 bp, Artificial Sequence
  Primer
ccgtgattatctctgtcagc

SEQ ID NO: 12 - DNA, 18 bp, Artificial Sequence
  Primer
ttccactctatcctctgc

SEQ ID NO: 13 - DNA, 18 bp, Artificial Sequence
  Primer
acttccgtccgatcatgg

SEQ ID NO: 14 - DNA, 18 bp, Artificial Sequence
  Primer
gctcgatcatctgcttcg

SEQ ID NO: 15 - DNA, 23 bp, Artificial Sequence
  Primer
gcgtggaccgcttgctgcaactc

SEQ ID NO: 16 - DNA, 18 bp, Artificial Sequence
  Primer
gctttcgatggcttcttc

SEQ ID NO: 17 - DNA, 19 bp, Artificial Sequence
  Primer
aactgaaaggcagaagttg

SEQ ID NO: 18 - DNA, 28 bp, Artificial Sequence
  Primer
gcgcatatggtttctatggatggatctc

SEQ ID NO: 19 - DNA, 26 bp, Artificial Sequence
  Primer
cgctcgagttatgcttgaagataacc

SEQ ID NO: 20 - DNA, 28 bp, Artificial Sequence
  Primer
gcgcatatggccgttaacgtctctgctc

SEQ ID NO: 21 - DNA, 26 bp, Artificial Sequence
  Primer
gcggatccttagccctcaagataacc

SEQ ID NO: 22 - DNA, 29 bp, Artificial Sequence
  Primer
gcggatccttactcgttttgaaggaaacc

SEQ ID NO: 23 - DNA, 30 bp, Artificial Sequence
  Primer
gcgctcgagtccggtgaaacgaagcaaatc

SEQ ID NO: 24 - DNA, 31 bp, Artificial Sequence
  Primer
gcggatccttacatacaattacgtgattttg

SEQ ID NO: 25 - DNA, 28 bp, Artificial Sequence
  Primer
actagtttcagtagcaatctaagtatgg

SEQ ID NO: 26 - DNA, 30 bp, Artificial Sequence
  Primer
gaattctgcttgaagataacccttgttatc

SEQ ID NO: 27 - DNA, 28 bp, Artificial Sequence
  Primer
ctcgagtttggaggaggagactaacaac

SEQ ID NO: 28 - DNA, 30 bp, Artificial Sequence
  Primer
tctagagccctcaagataacctttgttttg

SEQ ID NO: 29 - DNA, 26 bp, Artificial Sequence
  Primer
actagtaaatctggtttacttcatgc

SEQ ID NO: 30 - DNA, 28 bp, Artificial Sequence
  Primer
ggatccctcgttttgaaggaaacctttg

SEQ ID NO: 31 - DNA, 29 bp, Artificial Sequence
  Primer
gtcgacccttacaggttttgacaacagtc

SEQ ID NO: 32 - DNA, 29 bp, Artificial Sequence
  Primer
gtcgaccatacaattacgtgattttgtgg

SEQ ID NO: 33 - DNA, 1800 bp, Artificial Sequence
  T-DNA insertion sites of APK mutant alleles
ttcctcattgcctaattaaaaacattcagacacaaatttagtttcttcttcttttggttctgtctgaatccaattttcttatattttatttcctccaccaccgtgagatatgatcgccgccggagctaaatcgctattagggctttcgatggcttctccaaaagggattttcgatagcaattcaatgagtaattcaagatctgttgttgttgttcgtgcttgtgtttctatggatggatctcaaactctgagtcataacaaaaatggatctattcctgaggttaaatccattaacggtcacacgggtatgtatcgttcttcctgatttttcccagaacctttgatttttttgccaattttttatggttttaattgtctaattaggacaaaagcaaggacctttgtctacggtcggaaactcgacaaatataaagtggcatgaatgttctgttgagaaagttgatagacagagattgcttgatcagaaaggatgtgtgatttgggtcaccggtcttagtggttcaggtttgtgttgaaacaagcttcaagtttggtttctgtgtctatgtgaaatctatctaatgtttctatggatttataaatttgtagggaagagtactttggcttgtgctttgaatcagatgttgtatcaaaaggggaagctttgttatattcttgatggtgataatgttaggcatggcttaaaccgtgatcttagctttaaagctgaggatcgtgcagagaatattcgtagagttggtgagctttaatctctttaatccaatattggttgttgttgttagctttatctttttgacaatgtgttgttaattgctctgtttaggagaggttgctaagctttttgcggatgctggaataatctgcattgcgagtttgatatctccttatagaacagatagggacgcttgtcgaagtttgctccccgagggagattttgttgaggtattgacaactaagagcttggttttgttgtctctttataatactgaagttttcaagatttgagtaaaagtttgttttgatcaaataggtgttcatggatgtaccgcttagtgtttgcgaggcgagggatccaaagggtctttacaagcttgctcgtgcaggaaagatcaaaggtatctctagaaatgcggttggtaattgtatgacctatcaatgagacaaccccaaatgacattatagtttgatgtgtttcttcaggttttaccgggatcgatgacccttacgagccaccattgaactgcgaggtaaatgtcaataatattggaaaacagaattggatgctttggtttttgatcttgatgctgagcttttttggtgtcattcacttgtcttaccttttgttctattttctttttgtcatgtgaagatttctctaggacgtgaaggaggaacttctcctatcgaaatggcggaaaaggtcgtcggatacttagataacaagggttatcttcaagcataacatacttctagatacacaaatttgttatctcacggtcttagttctattttacgagccaaagtataaaagtaagcaaatccatggctgttttatggaatcacaagttcgacaaaataaatataaatctcataatttttcttgtccctttttttttgtatcataaatcttgatgttatggttctgtatataatatgtgtgaaataaggttaagcctactaaccattttatatgatctataatacaatatactttgtatgcactaatgaaaccaagacttcatggcttatccaaaattagggacaaaatggttgtgatgt

SEQ ID NO: 34 - DNA, 1900 bp, Artificial Sequence
  T-DNA insertion sites of APK mutant alleles
cgtctttcttatacataaacttattttgtacgatcacataaaactgtaaacccccacacagttattggccgtcggattcaatggaaggattagctatcagagcatctcgaccgtcggttttctgttctattccaggtctcggcggcgattcccaccgaaaacctccaagtgacggtttcctcaagctgcctgcgtcgtctattccggcggacagccgaaaattagtagcgaattctacttcctttcatccaatctcagccgttaacgtctctgctcaagcttccctcaccgctgattttcccgccctttcaggttctcccatctctctctgtttcgtcatatagcagatcgttcacatttcagaaaaagcaatttaggtttaagcatctccacaaatcgtgttgttgtttttttctgaccatttttacaatctctttaactttgtgtttgcgcagaaactatactgaaagagggaagaaataacggaaaagagaaagcagagaacatcgtgtggcacgagagttcgatatgcagatgcgacagacaacaacttcttcaacaaaagggttgtgtcgtttggatcactggtctcagtggttcagggaaaagcactgttgcttgtgcactaagtaaagcattgtttgaaagaggcaaacttacttacacactcgacggcgacaatgtccgtcacggccttaaccgtgacctcactttcaaagcagagcaccgcaccgaaaacattagaagaattggtacacaatttttcctttcttccaaaaattataatcatgtgtttactaactcatgaaaccaatttctttaacgcgttttatggtatatgcttaatggtttttgtaaagtgatttgtgattgatctttaaatttaatatcaggtgaggtggctaagttgtttgctgacgtcggagtcatttgtatagcaagtttgatttctccgtaccggagagacagagacgcgtgccggtccttgttacctgacggcgatttcgtcgaggtacgtacgctataacgttttactatatatgaatttgcttgttatagaagatatattagtatacattgattacattacgaaaacttactttgatttaggtcttcatggacgttcctcttcatgtgtgcgagtcgagagatccaaaggggttgtacaagcttgcacgtgcaggcaaaatcaaaggtatcccaaaaaaaagaaaacgtacttgtataaaagttgactttgactttttattttgttcgattcgtagagtttgacttattgacctaattgttgagtgtaacttattgacctagctaattgttgagttttcttgaattaattatccaggcttcactggaatcgacgacccttacgaggcaccagtgaattgcgaggttcgtcacgtgaatgatcagatagattttggaacgtaaatttgtactaataaagtattgatgtaatatcctccaggtagtgctgaaacacacaggagacgacgagtcgtgttcgccacgtcagatggctgagaacatcatctcttacctgcaaaacaaaggttatcttgagggctaagtcaaagtcggaaaaatcaacccaatttgtcgattctatatatggctaaaggattgttctccctgattcggtcagaagcttgagacctggaaacgatgtcgtctcgcttcgtcacactatcatttgttcttgaattgtgttgaagtcaaaagccaaagggaatatatctatatttgttctaatcatcatttagttagtttataaagagtttttgctccaatgatatcgactgtgttgtataatgtgaattaataaagtggatttcgttgtatttaagtttgtcttaccagcctaactaaattatgtgatttggactcatgaacccgttga

SEQ ID NO: 35 - DNA, 1700 bp, Artificial Sequence
  T-DNA insertion sites of APK mutant alleles
attctcttggtcttccagtttatatttatggaggaacatagtctcattaacaagcttgcattgttctgttccattggattgttataaaaatattcattggtcaattgagaaatcagtgtctaattttttttgaacgtgtctttgcagattgaatgctgtttctggtggattagtttctctgagttttggtgatagatcagattaacaggcagaattttgagaatgtcgacagtgggaaattcaacgaacatattttggcaagaatcccccattgggaaaactgaaaggcagaagttgttaaatcagaagggttgtgtggtgtggatcacagggctcagtggctcaggtcaatcttctaaaaaatcttaaattcttttcccattgtgatacttacttaagatgccttctttatatctctgtctatattctaaaatcttagcctcaaactttgtgcagggaaaagcacgttagcttgctcgcttagtagagagctgaacaaccggggaaagctatcatatattcttgatggtgacaatcttcgtcatggtttgaacaaagatcttggtttcaaggcagaggatagagtggaaaatatacgcagggtcggtaagtagtacatagttgcttactatcttttgctttcagttgatgaaagtctgattccgaagaaactcactaatacaaatgcaaatcctttaaaaaggtgtctcatatgtagtgatagtttacatggtaccaatacctcattgatatacatattggtatacacctaaaagctgatagattctggttaatgttgaaagaaacttctctaaagggcaaaatactaaagaagaccaacaatttgttctaaatgcaggagaagtagccaaactctttgcggatgctggtttaatctgtattgccagcctcatatccccgtatagaaaagaccgtgacgcctgcagggaaatgatacagaattcatcttttattgaggcaagctattcaaaaccaggctttacagtttgtaacccccattcggttggatcttaaaaacatcttgtttgatattgtggccaaatttgttaggttttcatgaacatgtctctgcaattgtgtgaagcaagggaccctaaaggcctatacaagcttgcacgtgcaggaaagatcaaaggtgagataacatagcttcaaggttccatataaacagaagatagcaagttgcttcaatatcatttgagttgttctataaaagtttttttttcttcatacccttttgtgtaggtttcacaggaatagatgatccgtatgagtctcccttgaactgtgaggtatgtgatccatgtgaacataagattcttcttcaagaatattgcaaacaacgcgtatgtgaatacgtttctctgtttcttttgtatgttttgaaacataacagatagagttgaaagagaaagagggagagtgtccttcccctgtagctatggctgaggaagtgatctcttatctagaagacaaaggtttccttcaaaacgagtaacgtccatcataatctaacgaccgagatttgtttttccttttactttttatgggtgttctctgtttgttactttcctgaaacactgcaaattcttaagaagtttcaactaatgaagacatatatagcttcttctccttcttctgaaacttgtcttctacactgaaactctagtcagtcaata

SEQ ID NO: 36 - DNA, 1900 bp, Artificial Sequence
  T-DNA insertion sites of APK mutant alleles
attcgagaatctcactaaacaaatagattaggtgaatccgacaaaagccaatgatgccgtcacgctcataaaactcggattccacaagcccaaaatagacaacatgttttattttattatttgtttaccgacttaaaatattaatttaagatcgttaaaaaacaaactctacaagtttttgatttcagagtccaccgttgagcctgttcaattaaatcgttgaatctcgcggtgcgttttactttgattctttcgaattttttttatatggatgttgccgcgatggcgagatgtgtgggaaggtgctacgtttcgccggcgttcggtgagtctgaatcgcaccggttatcggagcggcggtttctgaaattgtctagctccacgaattcggatcccgccggtagtaaaagtttgaagcttcgcgggaaaattcatcggagaatgagctacttccgtccgatcatggcaaaagatgaatccatttcatcgcggtccggtaataatcacgtaattccgttctggttcatctctaatcctagtttgggttcctatgattcgtttttaacatttgggttcctgagaccagaaatttccatagttttagcataaagtttggaccttagtagacagatcaatgatgtatatctgaatagatctaatctcagataatctaaccaggtgaaacgaagcaaatcaatggaaagcaaaagaacattgtctggcatgattgtcccgttactaaatccgacaggcaagaattaattaagcaaaagggatgtgtgatttggattactggcttaagtggttcaggtttggtcctttgtttctgaattgatctctaactacatcttgttatgtttctactggattctttcaaatattctcgtattttttcaggtaaaagtagtctggcatgtgctcttagtcgagctttgcacaatcgtggaaagctttcgtatatacttgatggtgacaatgttcgacatggtttaaacagcgatcttagtttcgaagcagatgatcgagctgaaaacattcgaagagttggtgaagtggctaaactgtttgcagattctggtattatctgtattgcaagtttaatatctccttaccggatagaacgagctgcctgccgtgcattattaccacaaggagatttcattgaggtttctccaagttcatgaatttgaatcccagtctcaacatcaatctgactttctcttggttctcttgataggtatttatggatgtgccactccatgtttgtgaagctagagatccaaagggcttatacaaacgtgcacgcgctggtaaaatcaaaggtaactttgctttttctctgcatctgttttcaacttaacccatgttctactgattttcattaattatataaccttcttgatctatccatttgattatatgtaaatatattgttccccaaaacatttcttctccaggttttacaggagtagatgatccatatgaagcgcctttggattgcgaggttcatataatatccaatttttgttatataagttggaatctagctcatctccgaacttgaatctaatgttgtgtttccgactactctttgcagattgtaatacaaaacagtagagacaaggggctttcttcttcatcttcatcttcatcttcaccttcatcttcgtcttcttctctgtgtgaaatggcagatattgttgtgtcgtacttggaccaaaatggatacctgaagaaacactccacaaaatcacgtaattgtatgtaaatgtaatattaatatcattgttgtaacgtttcttaaacctattttacctatggacgattaaattgtgaaaacataaatgtaattcacaatctgcaaagagttggtaagaaaaacttgtttgattgattgaga

(19) United States
(12) Patent Application Publication
(10) Pub. No.: US 2010/0287663 A1
(43) Pub. Date: Nov. 11, 2010

(54) METHODS FOR PRODUCING PLANTS WITH ALTERED LEVELS OF SULPHATED SECONDARY METABOLITES

(75) Inventors: STANISLAV KOPRIVA, COLNEY (GB); SARAH G. MUGFORD, COLNEY (GB)

Correspondence Address: MCKEE, VOORHEES & SEASE, P.L.C., 801 GRAND AVENUE, SUITE 3200, DES MOINES, IA, 50309-2721, US

(73) Assignee: PLANT BIOSCIENCE LIMITED, NORWICH (GB)

(21) Appl. No.: 12/437,234

(22) Filed: May 7, 2009

(51) Int. Cl. A01H 1/00; C12N 15/82; A01H 5/00; A01H 5/10; C12N 5/04

(52) U.S. Cl. 800/279; 800/284; 800/298; 800/301; 800/306; 435/410; 435/468; 435/440

(57) ABSTRACT

According to the present invention there is provided a method of producing a plant or a plant cell having an altered level of at least one glucosinolate, said method comprising obtaining a plant or plant cell comprising at least one active Adenosine-5′-Phosphosulphate Kinase (APS kinase, Apk) gene and preventing or inhibiting the production and/or function of the gene product encoded by at least one Adenosine-5′-Phosphosulphate Kinase (APS kinase, Apk) gene, preferably disrupting expression of at least one Adenosine-5′-Phosphosulphate Kinase (APS kinase, Apk) gene.

### FIELD OF THE INVENTION

The invention relates to methods for the production of plants and plant cells having altered levels of glucosinolate and to plants and plant products produced by said methods.

### BACKGROUND OF THE INVENTION

Sulphur is an essential nutrient for plant growth. Plants meet their demand for sulphur by assimilation of inorganic sulphate (FIG. 1). Sulphate is taken up via sulphate transporters and incorporated into adenosine 5′-phosphosulphate (APS) by ATP sulphurylase (ATPS). APS can then be used in two ways: it can be sequentially reduced by APS reductase and sulphite reductase to sulphite and sulphide, and incorporated into O-acetylserine to form cysteine (for a recent review see Kopriva 2006). APS can also be phosphorylated to 3′-phosphoadenosine 5′-phosphosulphate (PAPS) by the action of APS kinase (Apk). PAPS is the sulphate donor for the creation of sulphated secondary metabolites, which are formed by sulphotransferase (SOT) enzymes. The SOTs transfer the sulphate group from PAPS onto free hydroxyl groups of appropriate acceptor molecules. Sulphated plant metabolites include glucosinolates, phytosulphokines, and some hormones and flavonoids.

The glucosinolates are a large group of sulphur-rich amino acid-derived metabolites, found mainly in the order Capparales (Halkier & Gershenzon, 2006; Fahey et al., 2001). Within the Capparales are the Brassicaceae, which contain many agriculturally important food crops such as cabbage, broccoli, cauliflower, oilseed rape and also the model plant Arabidopsis thaliana. Glucosinolates are an important form of defence against herbivore and insect attack since their breakdown products are both toxic and deterrent to the attacker (Halkier & Gershenzon, 2006). The glucosinolates are also important nutritionally, as their breakdown products have been shown to have anticarcinogenic activity in humans (Mithen et al., 2003). They can however have a negative impact when present in high amounts in animal feed derived from oil seed rape (Schöne et al., 1997). Rapeseed varieties with low seed glucosinolate content have been developed based on alleles from a Polish variety “Bronowski” (Hasan et al., 2008). The result was the release in 1974 of the first 00-quality spring rapeseed variety, “Tower”. Today the majority of modern oilseed rape varieties have 00-quality. However, residual segments of the “Bronowski” genotype in modern cultivars are believed to cause reductions in yield, winter hardiness, and oil content (Sharpe and Lydiate, 2003). Furthermore, the restricted genetic variability in modern 00-quality oilseed rape (Hasan et al. 2006) is particularly relevant with regard to the development of genetically diverse heterotic pools of adapted genotypes for hybrid breeding. Thus, new approaches to reduce glucosinolates in rape seeds are needed to overcome this limitation. Glucosinolate biosynthesis and its regulation have been well-studied in Arabidopsis (for reviews see Yan & Chen, 2007; Halkier & Gershenzon, 2006; Grubb & Abel, 2006). The biological activity of glucosinolates depends on the sulphate group (Ratzka et al., 2002). The sulphation of the desulpho-glucosinolates (ds-gls) precursors is the final step of glucosinolate synthesis (Underhill et al., 1973). In Arabidopsis, the group VII SOTs AtSOT16, 17 and 18 are responsible for the sulphation of ds-gls (Piotrowski et al., 2004; Klein et al., 2006).

Other plant metabolites which depend on PAPS for sulphation include hormones such as 12-hydroxyjasmonate and 24-epibrassinolide. The sulphated derivatives of these have been detected in plant tissues, and in Arabidopsis the corresponding sulphotransferases have been identified (Gidda et al., 2003; Rouleau et al., 1999). Sulphation of these hormones is believed to modulate their biological activity, but the precise roles of these sulphated derivatives have yet to be elucidated. Plants also produce at least two classes of small sulphated peptides (Matsubayashi & Sakagami 1996; Amano et al., 2007). Phytosulphokines (PSK) are sulphated pentapeptides that stimulate cell proliferation in culture after binding to a membrane localised receptor (Yang et al., 2001; Matsubayashi et al., 2006). Disruption of this receptor leads to premature senescence of rosette leaves and gradual loss of the ability to form calluses (Matsubayashi et al., 2006). Another sulphated oligopeptide named PSY1 was recently isolated from Arabidopsis and its receptor was identified (Amano et al., 2007). Plants that lost the ability to bind PSY1 due to disruption of three paralogous receptor kinases displayed a semi-dwarf phenotype again with a premature senescence. Both peptides require tyrosine sulphation for biological activity (Matsubayashi et al., 1996; Amano et al., 2007).

Despite the wide range of sulphated compounds in plants, very little is known about the enzyme providing the activated sulphate donor PAPS, the APS kinase (“Apk”). Previous studies have identified two functional Apk isoforms in Arabidopsis (Lillig et al., 1998; Lee & Leustek, 2001). The completion of the Arabidopsis genome sequence has revealed a further two predicted isoforms based on sequence homology to the original members (The Arabidopsis Genome Initiative, 2000).

The inventors have localised and identified the function of these four Apk isoforms in Arabidopsis. They have shown that Arabidopsis possesses four functional isoforms of Apk and that the Apk1 and Apk2 isoforms play not only a major role in control of synthesis of sulphated metabolites but are important for normal vegetative development. These observations will apply with equal force to other members of the Brassicaceae.

### SUMMARY OF THE INVENTION

The invention is based on the inventors' identification and characterisation of the Apk isoforms and plants with altered levels of Apk.

According to a first aspect of the present invention there is provided a method of producing a plant or a plant cell having an altered level of at least one glucosinolate, said method comprising obtaining a plant or plant cell comprising at least one active Adenosine-5′-Phosphosulphate Kinase (APS kinase, Apk) gene and preventing or inhibiting the production and/or function of the gene product encoded by at least one Adenosine-5′-Phosphosulphate Kinase (APS kinase, Apk) gene.

As used herein, the term “gene product” refers to a polypeptide (or a fraction or precursor thereof) encoded by a gene said polypeptide having an APS kinase activity.

As used herein, the term “preventing or inhibiting the production of the gene product” means preventing or inhibiting production of a functional polypeptide by any means known in the art. The production or function may be fully or partially prevented. In one embodiment, preferably the production or function of the gene product is fully prevented, i.e. there is no active gene product. In some instances the production or function of the gene product may be disrupted such that there is only about 5%, about 10% about 20%, about 30%, about 50%, about 60%, about 70%, about 80%, about 90% or about 95% of the wild type level of expression remaining.

By the term “preventing or inhibiting the production of the gene product encoded by at least one Adenosine-5′-Phosphosulphate Kinase (APS kinase, Apk) gene” as used herein it is meant that the production of the gene product may be prevented or inhibited by (a) knocking out said gene in said plant and deriving a progeny plant homozygous in said knockout; (b) post-transcriptionally silencing said gene through the use of iRNA or antisense RNA (gene silencing); (c) transcriptionally silencing said gene by, for example, epigenetic techniques; (d) preventing or altering the function of the gene product by the introduction of at least one point mutation by, for example, TILLING; (e) post translationally inactivating the gene product.

It will be apparent that the methods by which the production of the gene product may be prevented or inhibited are those known to those skilled in the art and include: (a) knocking out said gene in said plant and deriving a progeny plant homozygous in said knockout; (b) post-transcriptionally silencing said gene through the use of iRNA or antisense RNA (gene silencing); (c) transcriptionally silencing said gene by, for example, epigenetic techniques; (d) preventing or altering the function of the gene product by the introduction of at least one point mutation by, for example, TILLING; (e) post translationally inactivating the gene product.

According to a second aspect of the present invention there is provided a method of producing a plant or a plant cell having an altered level of at least one glucosinolate, said method comprising obtaining a plant or plant cell comprising at least one active Adenosine-5′-Phosphosulphate Kinase (APS kinase, Apk) gene and disrupting expression of at least one Adenosine-5′-Phosphosulphate Kinase (APS kinase, Apk) gene.

According to a third aspect of the present invention there is provided a method for altering the level of at least one plant glucosinolate which method comprises obtaining a plant which comprises at least one active Adenosine-5′-Phosphosulphate Kinase (APS kinase, Apk) gene and rendering at least one Adenosine-5′-Phosphosulphate Kinase (APS kinase, Apk) gene non-functional.

By the term “altered” or “reduced” or “increased” herein (for example with regard to glucosinolate level and/or expression level) we mean altered, reduced or increased compared with the level in a wild type plant or plant cell. The term “wild type plant or plant cell” as used here in means a plant or plant cell which is otherwise identical to the plant or plant cell according the present invention except that the expression of the at least one Adenosine-5′-Phosphosulphate Kinase (APS kinase, Apk) gene has not been disrupted.

It will be further apparent that the “altered level” refers to the level of functional or active glucosinolate. Therefore, included within the scope of the invention is the situation in which high level of non-functional or inactive glucosinolate derivatives are produced.

As used herein, the term disrupting expression of the gene means preventing expression of the gene by any means known in the art. The expression may be fully or partially prevented. In one embodiment, preferably the expression of the gene is fully prevented, i.e. there is no expression of the gene. In some instances the expression of the gene may be disrupted such that there is only about 5%, about 10% about 20%, about 30%, about 50%, about 60%, about 70%, about 80%, about 90% or about 95% of the wild type level of expression remaining.

By the term “disrupting expression of at least one Adenosine-5′-Phosphosulphate Kinase (APS kinase, Apk) gene” as used herein it is meant that the production of the gene product may be prevented or inhibited by (a) knocking out said gene in said plant and deriving a progeny plant homozygous in said knockout; (b) post-transcriptionally silencing said gene through the use of iRNA or antisense RNA (gene silencing); (c) transcriptionally silencing said gene by, for example, epigenetic techniques; (d) preventing or altering the function of the gene product by the introduction of at least one point mutation by, for example, TILLING.

It will be apparent that the methods by which the gene may be disrupted are those known to those skilled in the art and include: (a) knocking out said gene in said plant and deriving a progeny plant homozygous in said knockout; (b) post-transcriptionally silencing said gene through the use of iRNA or antisense RNA (gene silencing); (c) transcriptionally silencing said gene by, for example, epigenetic techniques; (d) preventing or altering the function of the gene product by the introduction of at least one point mutation by, for example, TILLING.

By appropriately knocking out specific Apk genes, as disclosed herein, it is possible to alter the level and/or composition of glucosinolates produced by the plant or plant cell. Furthermore, by careful selection of which genes are rendered inactive and/or of the method of inactivation, it is possible to tailor the nature or level of glucosinolates produced in different parts of the plant. This is due to the differential expression of different Apk genes in different plant tissues or plant organs, as discussed below.

As used herein, the term gene knockout refers to a method of abolishing the activity of a pre-selected gene, e.g. Apk gene, by insertion of one or more nucleotides into the gene or its promoter, and/or deletion of one more nucleotides from the gene or its promoter such that the product of the gene is not expressed. Techniques for achieving this are well known in the art.

As used herein the term iRNA refers to RNA interference (RNAi). This is a method of post-transcriptional gene silencing (PTGS) in eukaryotes induced by the direct introduction of dsRNA (Fire A, et al., (1998)).

In some embodiments, plants having reduced expression and/or function of the APK-related polypeptide may be produced by random mutagenesis, followed by screening of mutants for reduced APK-related polypeptide expression. Suitable techniques are well known in the art and include Targeting Induced Local Lesions IN Genomes (TILLING).

TILLING is a high-throughput screening technique that results in the systematic identification of non-GMO-derived mutations in specific target genes (Comai and Henikoff, The Plant Journal (2006) 45, 684-694 Till et al BMC Plant Biol. Apr. 7, 19, 2007).

Those skilled in the art will also appreciate that, based on the genetic information disclosed herein, Targeted Induced Local Lesions IN Genomes (“TILLING”, e.g. utilizing PCR-based screening of plants generated through chemical mutagenesis (generally via ethyl methane sulfonate (EMS) treatment), often results in the isolation of missense and nonsense mutant alleles of the targeted gene(s); TILLING permits the high-throughput identification of mutations in target genes without production of genetically modified organisms and it can be an efficient way to identify mutants in a specific gene that might not confer a strong phenotype by itself), may be carried out to produce plants and offspring thereof with a change in the APK gene(s), thereby permitting identification of plants with specific phenotypes relevant to plant sulphur and glucosinolate metabolism.

As used herein the term “TILLING” (Targeting Induced Local Lesions in Genomes) refers to a method that allows directed identification of mutations in a specific gene. The method combines standard mutagenesis with a chemical mutagen such as ethyl methane sulfonate (EMS) and DNA screening-techniques that identify single base mutations (also called point mutations) in a target gene (see for example McCallum et al., 2000, Colbert et al.,2001, incorporated herein by reference).

It will be further apparent that the term “altered level of at least one glucosinolate” as used herein can refer to an increase or a decrease in the level of glucosinolate present in the plant or plant cell. This may be the total glucosinolate level present in the plant (i.e. in all tissues or organs of the plant) or plant cell. Alternatively or in addition thereto this may be the glucosinolate level in a specific tissue or organ of the plant, for example the leaves and/or the seed and/or inflorescences and/or roots. Preferably, the total level of glucosinolate in the plant or plant cell is reduced. More preferably, the level of glucosinolate in one or more specific tissues or organs (e.g. seed and/or leaves and/or inflorescences and/or roots) of the plant is reduced. The level of glucosinolate in a specific tissue or organ (e.g. seed and/or leaves and/or inflorescences and/or roots) or the total level of glucosinolate in the plant or plant cell may be reduced by at least 10%, 20%, 30%, 40%, 50%, 60%, 70%, 80%, 90%, or 100%.

The glucosinolate level may be a single glucosinolate or more than one glucosinolate (e.g. a mixture of glucosinolates). In some embodiments the glucosinolate may be one or more aliphatic glucosinolate (for example 4-methylsulphinylbutyl-gls (4MSOB)) and/or one or more indolic glucosinolate (for example I3M, 4MOI3M).

It will be understood that a reduction in the total level of glucosinolate of 100% results in a plant which expresses no glucosinolate in its tissues or organs.

In one preferred embodiment, the level of glucosinolate in different specific plant tissues or organs may be differentially altered. In other words, the amount of glucosinolate present in different tissues or organs of the plant produced by the present method may vary.

For example the leaves of a plant produced in accordance with the present invention may comprise more glucosinolate than the seeds and/or inflorescences and/or roots of said plant. It is known that levels of glucosinolate vary naturally within plant tissues and are linked to levels of expression of Apk. In leaves the highest levels of Apk and glucosinolate are seen around the vasculature and the outer lamina (Shroff et al. 2008). This is believed to be due to the fact that these are the regions most vulnerable to damage by insects. Furthermore, it has been suggested that glucosinolate is not produced in the seeds of plants, but is transported there from other regions of the plant in the phloem. The source region for this seed glucosinolate may be for example, the leaf or silique.

Preferably, the leaves of the plant maintain a protective effect against pests or infection similar to that of the wild type plant.

It will be understood that the protective effects seen in the leaves may be due to the remaining glucosinolate or due to the increased levels of other compounds which provide a protective effect against pests.

These compounds may be for example thiol compounds, phytoalexin, camalexin and glutathione which may represent alternative protection against pests.

More preferably, the level of glucosinolate present in the seed and/or inflorescences and/or roots is reduced to a level where no adverse effects (such as palatability to livestock) due to its presence are seen in products produced from the seed and/or inflorescences and/or roots, e.g. oil and/or meal and/or feedstuff.

It will be understood that plants or plant cells according to the invention having a reduced protective effect against pests or infection compared to the wild type plant may still be useful if the protective effect is still sufficient to deter pests sufficiently for the plant to be commercially useful.

In one embodiment the level of glucosinolate present in the seed and/or inflorescences and/or roots (i.e. the overall level of glucosinolate in the seed, inflorescence, root—which may be more than one glucosinolate) is less than the level of glucosinolate present in the leaves (i.e. the overall level of glucosinolate in the leaves—which may be more than one glucosinolate).

It will be further apparent that the plant or plant cell can be produced by any means known in the art, for example, the use of recombinant technology followed by regeneration of an adult plant using standard molecular biological techniques.

Preferably, the plant or plant cell is a member of the family Brassicaceae more preferably a member of the genus Brassica. The Brassica include many commercially important species such as B. carinata—Abyssinian Mustard or Abyssinian Cabbage, used to produce biodiesel; B. elongata—Elongated Mustard; B. fruticulosa—Mediterranean Cabbage; B. juncea—Indian Mustard, Brown and leaf mustards, Sarepta Mustard; B. napus—oil seed rape, canola, Rutabaga (Swede Turnip); Nabicol; B. narinosa—Broadbeaked Mustard; B. nigra—Black Mustard, B. oleracea—kale; cabbage, broccoli, cauliflower, Kai-lan, Brussels sprouts; B. perviridis—tender green, mustard, spinach; B. rapa (syn B. campestris)—Chinese cabbage, turnip, Rapini, Komatsuna; B. rupestris—Brown Mustard; B. septiceps—Seventop Turnip; B. tournefortii—Asian Mustard, as well as the model organism Arabidopsis thaliana.

In a preferred embodiment, the plant or plant cell is oil seed rape or canola.

It will be understood by the skilled person that many species of plant comprise more than one Apk gene. Thus, it will be understood that the level of glucosinolate present in a plant or plant cell may not be directly related to the expression of one particular Apk gene. If this is the case, disrupting expression of a single Apk gene may have no, or very little, effect on the overall level of glucosinolate present in a plant or plant cell. This is due to the complementary nature of the Apk genes (i.e. functional redundancy) present in some plants. If a plant or plant cell comprises more than one Apk gene, these may be complementary.

By the term “complementary nature” used herein we mean that for example when two genes (e.g. Apk genes) are expressed in a plant or plant cell and the expression of one of these genes (e.g. one of these Apk genes) is disrupted, the level of glucosinolate in the plant, plant cell or specific plant tissue or plant organ is not (or substantially not) reduced. Thus these genes are considered to be complementary in nature and for the purposes of the present invention both genes would need to be disrupted in order to alter the level of glucosinolate in the plant, plant cell, plant tissue and/or plant organ. In some instances, more than two genes may be complementary in nature, e.g. three or four genes.

Therefore, in a preferred embodiment, when said plant or plant cell comprises more than one Apk gene, expression of the gene product of at least two Apk genes may be disrupted, e.g. two, three or four Apk genes.

Preferably, the method comprises the further step of determining which Apk genes in said plant are complementary and disrupting expression of said complementary genes.

It will be apparent that the complementary genes can be identified by creating Apk double knock-out mutant plants or plant cells.

By “double knock-out mutant” we mean plants or plant cells in which the expression of two Apk genes has been disrupted by insertion and/or deletion of one or more nucleotides from the gene and/or promoter.

These are created by crossing homozygous single knock-out mutant plants having knock-out mutations in different, Apk genes. The presence of the double mutant can be confirmed, for example, through PCR genotyping of the Apk loci. Complementary genes can be identified by analysing the levels of glucosinolate in the double mutants. Those plants comprising significantly less glucosinolate than the wild type plants can be seen to comprise mutations in complementary genes.

In one embodiment, the present invention provides a method of producing a plant or plant cell having an altered level of at least one glucosinolate, said method comprising:
a) obtaining a plant or plant cell comprising at least one active Apk gene;
b) identifying the complementary Apk genes in a plant or plant cell; and
c) disrupting expression of said complementary Apk genes such that the level of at least one glucosinolate is altered (preferably reduced) in the plant, the plant cell, a plant tissue or a plant organ (e.g. the seed, inflorescence or root).

Preferably, the method comprises the step of determining which of said Apk genes correspond in function and/or sequence to Arabidopsis Thaliana Apk1 and Apk2 and disrupting expression of the gene products of said genes.

Preferably, said Apk genes have at least 50%, 60%, 70%, 75%, 80%, 90%, 95%, 98%, 99% sequence identity with the DNA sequence of Apk1 and/or Apk2 from A. thaliana.

Preferably, the polypeptides encoded by said Apk genes have at least 30%, 40%, 50%, 60%, 70%, 75%, 80%, 90%, 95% homology to the polypeptides encoded by Apk1 and/or Apk2 from A. thaliana.

In one embodiment of the current invention, the disruption of the Apk gene product occurs post transcriptionally. Preferably, said disruption is effected by iRNA. It will be understood that said plant or plant cell can be transformed with a plasmid which expresses the iRNA under the control of a promoter and that said promoter can be constitutive or tissue specific.

It will further be apparent to the skilled person that due to homology between Apk isoforms, it may be possible to use a single iRNA construct to target more than one Apk transcript.

It will be understood that the skilled person will be aware of plant transformation and regeneration techniques and of suitable plasmids and promoters for expressing iRNA in plant cells.

Examples of tissue specific promoters suitable for use in the present invention include APK1, LeB4, SULTR1;2, STLS1, AAP2, SHATTERPROOF1 and SEEDSTICK. These are further described in Table 8 below. Other tissue specific promoters are known, for example, US2008/0244791 (incorporated herein in its entirety) discloses root specific promoters.

To reduce gls synthesis specifically in the tissue producing gls for transport into seeds the iRNA is expressed under the control of the corresponding tissue specific promoter.

In an alternative embodiment, the disruption of the Apk gene product occurs at the transcriptional level. Preferably, said disruption is effected by insertion of at least one nucleotide into an Apk gene (knock-out mutation) or deletion or at least one nucleotide into an Apk gene (knock-out mutation).

It will be understood that the skilled person will be aware of techniques for producing knock-out mutations of genes, for example, the use of integrating viral vectors or transposons.

In a further embodiment, the disruption of the Apk gene product is effected by introduction of at least one point mutation.

It will be understood that the skilled person will be aware of techniques for producing point mutations in genes, for example, the use of TILLING techniques.

According to a fourth aspect of the present invention there is provided a method of producing a genetically transformed plant or plant cell having an altered level glucosinolate, comprising the steps of:
i) identifying the presence of at least one active Apk gene in a plant or plant cell,
ii) disrupting the expression of at least one Apk gene.

According to a fifth aspect of the present invention there is provided a method of altering the level of at least one glucosinolate in a plant or plant cell comprising disrupting the expression of at least one Apk gene in said plant or plant cell.

It will be apparent to a skilled person that Apk genes from a plant species of interest can be identified by homology searching of databases for sequences homologous to Apk from A. thaliana, or known Apks from other species. These searches can be carried out using, for example, BLAST searching (www.ncbi.nlm.nih.gov/BLAST/) or other available databases.

If no sequences homologous to Apk can be identified by database searching, Apk genes can be identified using standard molecular biological techniques such as cloning known to those skilled in the art and disclosed in, for example, Sambrook et al. (2001) Molecular Cloning A Laboratory Manual, (Third Edition) CSHL Press, incorporated herein by reference in its entirety.

Preferably, all Apk genes from the plant species of interest are identified via homology searching/cloning and an expression profile of the Apk gene products in different tissues or organs is produced using standard techniques known to the skilled person.

In one preferred embodiment, the disruption of the gene product of the at least one Apk gene occurs at the RNA level. Preferably, disruption is effected by iRNA.

Preferably, the plant or plant cell is transformed with a plasmid encoding an iRNA under control of a promoter. It will be apparent that this promoter may be a constitutive promoter and/or a tissue specific promoter.

In a further preferred embodiment said disruption of the gene product of the at least one Apk gene occurs at the DNA level. Preferably, said disruption is effected by insertion of at least one nucleotide into the Apk gene or deletion of at least one nucleotide from the Apk gene.

In a further embodiment, the disruption of the Apk gene product is effected by introduction of at least one point mutation.

It will be understood that the skilled person will be aware of techniques for producing point mutations in genes, for example, the use of TILLING techniques.

In one embodiment, said plant is regenerated from a plant cell produced according to said method.

In a preferred embodiment, the level of at least one glucosinolate in said plant or plant cell is reduced.

In one preferred embodiment, the level of glucosinolate is differentially altered in different tissues or organs in said plant. Preferably, the level of glucosinolate is higher in the leaves of the plant than the seeds inflorescences and/or roots. More preferably, the leaves retain protection against pests such as herbivores, e.g. leaf eating insects or infection by, for example, fungi.

It will be understood that the plant can be directly produced by said methods, can be regenerated from a plant cell according to said methods or can be produced from the seed of a plant produced by said methods.

It will be understood that a plant produced by the methods disclosed herein can be crossed with a further variety of the same species to produce new varieties of plant having reduced levels of glucosinolate either throughout the plant or in specific tissues and/or organs. It will be understood that this will be a useful tool for increasing the genetic variability of crops.

For example, the restricted genetic variability in modern 00-quality oilseed rape (Hasan et al. 2006) is particularly relevant with regard to the development of genetically diverse heterotic pools of adapted genotypes for hybrid breeding. The present invention may overcome this limitation.

According to the present invention there is also provided a method of producing a progeny plant comprising crossing a plant according to the present invention with another plant.

It will be understood that such a progeny plant may have increased genetic variability.

Also provided is a progeny plant produced by said method.

Also provided is a seed produced from such a progeny plant.

Preferably, said plants are oil seed rape or canola.

Also provided is a product, e.g. an oil, feedstuff, or a meal for example, produced from a plant produced according to the methods of the present invention.

A seed produced by a plant of the present invention.

A product (e.g. an oil, feedstuff, or a meal) produced from the seed of the present invention.

According to a further aspect of the present invention there is provided a method of creating an Apk double mutant, comprising the steps of:

crossing Apk single mutants;

segregating F2 seeds; and

screening said F2 seeds to identify homozygous double mutants.

By the term “double mutant” we mean wherein the expression of two Apk genes is disrupted.

By the term “single mutant” we mean wherein the expression of one Apk gene is disrupted.

It will be understood that preventing or inhibiting the production of the gene products of Apk genes or disrupting Apk genes may have an effect on the levels of sulphated compounds found in the plant in general. It will further be apparent that the levels of transcription of multiple genes may be affected and that these may either be up regulated or down regulated dependant upon the specific gene in question. It will be further understood that in many plant species, at least one of these sulphated compounds will be a growth factor.

According to a further aspect of the present invention there is provided method of producing a plant having a semi dwarf phenotype said method comprising obtaining a plant or plant cell comprising at least one active Adenosine-5′-Phosphosulphate Kinase (APS kinase, Apk) gene and preventing or inhibiting the production of the gene product encoded by at least one Adenosine-5′-Phosphosulphate Kinase (APS kinase, Apk) gene.

In one preferred embodiment, the Apk gene is disrupted.

As used herein, the term “semi dwarf phenotype” refers to a plant having a reduced size when compared to a wild type plant of the same species at the same stage of development.

The semi dwarf phenotype may result from a reduction in the level of at least one active compound whose sulphation is controlled by APS kinase. It will be understood that the at least one compound may be a growth factor.

In one embodiment, the growth factor is PSK.

The semi dwarf plants according to the present invention have an otherwise normal morphology when compared to wild type plants of the same species, for example, they flower and produce viable seed.

According to a further aspect there is provided a method of controlling the growth rate of plants comprising disrupting expression of at least one Adenosine-5′-Phosphosulphate Kinase (APS kinase, APK) gene.

Those skilled in the art will appreciate, based on the disclosure provided here, that various modifications, alterations or variations on the specifics described herein may be made without departing from the essence of the invention which is reflected in the appended claims.

### BRIEF DESCRIPTION OF THE DRAWINGS

FIG. 1. Sulphate Assimilation Pathways in Plants

Sulphate can be assimilated in a reduction pathway to cysteine (left) or via PAPS to sulphated compounds (right). The enzymes are shown in italics; Apk is shown in bold. SULTR, sulphate transporter; SiR, sulphite reductase; SAT, serine acetyltransferase; OASTL, O-acetylserine(thiol)lyase; PAP, adenosine-3′,5′-bisphosphate; R, acceptor molecule.

FIG. 2. Phylogenetic analysis of APS kinases from plants and green algae.

Neighbor-joining tree was constructed from alignment of amino acid sequences of APS kinases from A. thaliana (At), Oyza sativa (Os), Populus tremuloides (Pt), Selaginella moellendorffii (Sm), Physcomitrella patens (Pp), the green algae Chlamydomonas reinhardtii (Cr), Ostreococcus tauri (Ot) and Ostreococcus lucimarinus (01), and Escherichia coli (Ec) as an outgroup. Numbers represent results of bootstrap analysis with 100 replicates. Asterisks mark sequences possessing putative plastid targeting peptides.

FIG. 3. Tissue Specific Expression of APK Isoforms

DNA fragments encoding 3500 bp upstream of ATG to ATG were fused to the GUS coding sequence and stably transformed into Arabidopsis. Images are as follows: A, rosette leaves; B, roots; C, root tip; D, flower; E, pollen grains and F, developing seeds in siliques. Expression of Apk1 and -2 in the funiculus is indicated with arrows (fn).

FIG. 4. SALK T-DNA insertions in Apk genes

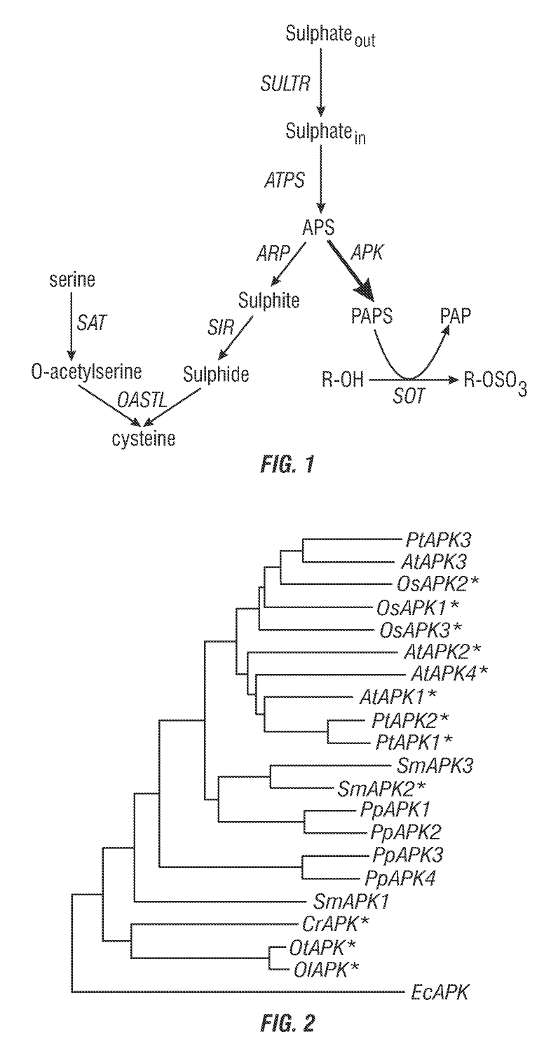

(A) The position of two independent SALK T-DNA insertion alleles for APK isoforms. Insertion sites are indicated by triangles. Exons are indicated by black boxes, introns as thin lines. For details of SALK alleles please see methods. (B) Confirmation of knockout status of plants homozygous for the T-DNA insertion by RT-PCR. cDNA from knockouts was amplified with primers specific to coding regions of APK isoforms, Col-0 is included as positive control. Note that the presence of contaminating genomic DNA is seen in apk3-2, this is due to the insertion being in the promoter thus allowing the amplification of a genomic fragment. (C) Total glucosinolate contents in 5-week-old leaves of the single knockout plants. Data represents mean ±SD for five replicates. (D) Sequence showing the positions of the insertion sites of the T-DNA.

FIG. 5. Creation and Confirmation of Double Mutants of Apk.

Pairs of single knockouts were used to create all six possible double knockout combinations. (A) PCR amplification of cDNA from homozygous double knock-out lines confirmed the lack of appropriate transcript in the mutants. (B) Growth phenotype of the apk1 apk2 mutant (right) compared to WT at 5 weeks in short days in controlled environment room. (C) Comparison of growth (based on rosette fresh weight) of apk1 apk2 double mutant plants with Col-0 during six weeks in short days. The data show the mean ±SD of 5 rosettes. (D) Total glucosinolate content in 5-week-old rosette leaves of the double knockouts. The data are presented as the mean of 5 replicates ±SD. Student's t-test significantly different to wild-type at **=p<0.01 and ***=p<0.001.

FIG. 6. Glucosinolate measurements in the apk1 apk2 double knockout.

Comparison of levels of sulphated glucosinolates in Col-0 and apk1 apk2 rosette leaves (A) and seeds (B). Levels of total, aliphatic and indolic glucosinolates (left graph) are presented along with the levels of individual aliphatic (middle graph) and indolic glucosinolates (left graph). 3MSOP, 3-methylsulphinylpropyl-GL; 4MSOB, 4-methylsulphinylbutyl-GL; 5MSOP, 5-methylsulphinylpentyl-GL; 6MSOH, 6-methylsulphinylhexyl-GL; 7MSOH, 7-methylsulphinylheptyl-GL; 4MTB, 4-methylthiobutyl-GL; 8MSOO, 8-methylsulphinyloctyl-GL; 7MTH, 7-methylthiobutyl-GL; 8MTO, 8-methylthiooctyl-GL; 40HI3M, 4-hydroxy-indolyl-3-methyl-GL; I3M, Indol-3-ylmethyl-GL; 4MOI3M, 4-methoxy-3-ylmethyl-GL; 1MOI3M, 1-methoxy-3-ylmethyl-GL. Data are presented as mean ±SD for four replicates. Student's t-test significantly different to wild-type at *=p<0.05, **=p<0.01, and ***=p<0.001. n.d.=not detected.

FIG. 7. Levels of ds-gls in Col-0 and apk1 apk2 Leaves and Seeds.

ds-gls precursors were quantified from leaves and seeds of Col-0 and apk1 apk2 plants. Each data point represents mean ±SD for four replicates.

FIG. 8. Levels of IAA in Apk Double Mutants

IAA content was determined from rosette leaves of 5-week-old Col-0 and all six double Apk mutants. The data are presented as means ±SD from four biological replicates. Asterisks mark values significantly different to wild-type at *=p<0.05, **=p<0.01, and ***=p<0.001.

FIG. 9. Expression of PSK precursor genes in apk1 apk2 double mutant.

cDNA was synthesised from RNA extracted from five week old rosette leaves of Col-0 and apk1 apk2 plants. Semi-quantitative RT-PCR was used to monitor the expression of phytosulfokin precursors PSK2, 3, and 4, and actin for normalisation.

FIG. 10. Levels of Thiols and APS in Apk Double Mutants

(A) cysteine and γ-EC and (B) glutathione content was determined in rosette leaves of 5-week-old Col-0 and all six double Apk mutants. The data are presented as means ±SD from three to four biological replicates. Asterisks denote values significantly (P<0.05) different from WT values.

FIG. 11. Insect choice bioassays using Col-0 and apk1 apk2 mutant plants.

Aphid bioassay: Pairs of Col-0 and apk1 apk2 plants were grown in caged pots for three weeks. The pots were infested with 10 Myzus persicae (green peach aphid). Numbers of insects per plant following 7d infestation were recorded. The numbers were corrected for leaf area. Data are mean ±SD of 30 replicates.
Sciarid fly: Feeding locations of Brassydia purperea (Sciarid fly) larvae were observed 7 days after infestation of plate-grown Col-0 and apk1 apk2 plants with sterile eggs. Data are the mean ±SD of 15 replicates.

Plutella: Plants were grown as for aphids and five leaf feeding Plutella xylostela (diamond back moth) larvae were added. Numbers of larvae per plant were recorded after 4 days. Data are the mean ±SD of 26 replicates.

### DETAILED DISCLOSURE OF THE PREFERRED EMBODIMENTS OF THE INVENTION

### Abbreviations

APS, adenosine-5′-phosphosulphate; ATPS, ATP sulphurylase; Apk, APS kinase; PAPS, 3′-phosphoadenosine-5′-phosphosulphate; SOT, sulphotransferase; GUS, β-glucuronidase; gls, (sulphated) glucosinolate; ds-gls, desulpho-glucosinolate; OPDA, 12-oxo-phytodienoic acid; JA, jasmonic acid, OH-JA, hydroxy-jasmonate; IAA, indole acetic acid; WT—wild type.

### 1. Arabidopsis has Four Functional APS Kinase Isoforms

Apk is positioned at a key branch of APS conversion which is directed to sulphur assimilation into reduced compounds or to organic sulphates. In order to determine the contribution of Apk to the control of synthesis of sulphated compounds, the inventors investigated the function, subcellular and tissue-specific localisation of the enzyme's isoforms, and the effect of disrupting the genes encoding them. All four Arabidopsis Apk isoforms were functional as assessed by analysis of the kinetic properties of the proteins following heterologous expression in E. coli. The comparison of reaction velocities and affinities to APS did not reveal any major differences among the recombinant proteins (Table 1).

Computer predictions suggested different subcellular localisations of the Apk isoforms (Table 1). This is in agreement with two sites of APS formation by ATPS, which is present in plastids and in the cytosol (Rotte and Leustek, 2000). It also reveals that there are two pools of PAPS in the cell, one in the plastid and one in the cytosol. The SOT enzymes which use PAPS as the sulphate donor for the sulphation of acceptor molecules to generate sulphated secondary metabolites, are, however, most probably cytosolic (Klein & Papenbrock, 2004), which is certainly the case for the ds-gls sulphating AtSOT16, 17, and 18 (Piotrowski et al., 2004; Klein et al., 2006). The function of the plastidic PAPS is thus not obvious, unless it is exported to the cytosol. This must clearly be the case since the apk3 mutants, which lack the only cytosolic Apk isoform, do not show any reduction in sulphated metabolites. PAPS transporters have been identified in Drosophila (Lüders et al., 2003; Kamiyama et al., 2003; Goda et al., 2006), and in mammals (Mandon et al., 1994). However, no PAPS transporter has been identified in plants so far.

TABLE 1
Features of Arabidopsis Apk isoforms

| | | TargetP | ChloroP | MW with | MW without | | |
|---|---|---|---|---|---|---|---|
| | | prediction/ | predicted | target | target | | |
| Isoform | AGIcode | Score | TP length | peptide | peptide | Km_{[APS]} | Vmax |
| Apk1 | At2g14750 | Chloroplast | 38aa | 29.8 kDa | 25.9 kDa | 5.3 μM | 20.1 units mg |
| | | 0.735 | | | | | protein⁻¹ |
| Apk2 | At4g39940 | Chloroplast | 59aa | 32.0 kDa | 25.8 kDa | 1.0 μM | 10.6 units mg |
| | | 0.548 | | | | | protein⁻¹ |
| Apk3 | At3g03900 | Other | — | n/a | 23.1 kDa | 7.4 μM | 12.4 units mg |
| | | 0.752 | | | | | protein⁻¹ |
| Apk4 | At5g67520 | Chloroplast | 75aa | 32.8 kDa | 26.3 kDa | 2.1 μM | 12.7 units mg |
| | | 0.653 | | | | | protein⁻¹ |

Since the presence of four Apk genes in Arabidopsis may be associated with the relatively high content of sulphated metabolites, i.e. the glucosinolates, in this species we explored the gene family in other plant species. Rice and poplar possess three Apk isoforms, as does the lycophyte Selaginella moellendorffii, while the moss Physcomitrella patens and the green algae Chlamydomonas reinhardtii and Ostreococcus tauri contain four and one Apk gene, respectively (Kopriva et al., 2007). Apk thus appears to be encoded by small multigene families in most plants analysed so far. There are, however, differences in the putative targeting of the derived proteins. While rice seems to encode only Apk isoforms with a chloroplast targeting peptide, in P. patens all Apk's appear to be cytosolic (FIG. 2). Interestingly, while rice and poplar genomes encode multiple SOT genes, no homologues to this class of sulphotransferases are present in P. patens or C. reinhardtii.

2. Apk Expression Co-Localises with Sites of Glucosinolate Synthesis

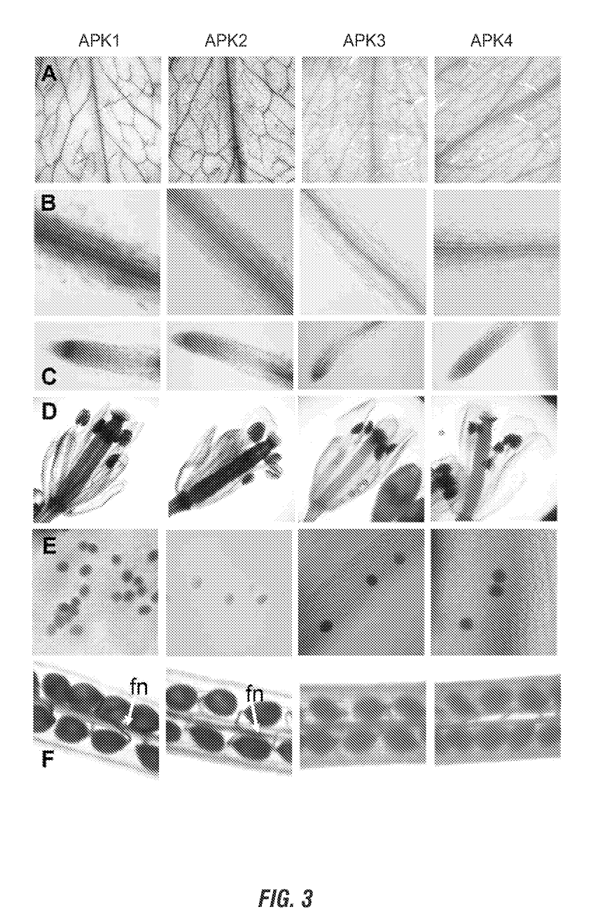

The analysis of tissue-specific expression of Apks using APKₚᵣₒ:GUS constructs revealed similarities but also striking differences between the isoforms (FIG. 3). Generally, in the roots and leaves, APK expression seems to be concentrated around the vasculature. Expression was more specific in the root tip, where all four isoforms showed different patterns of expression, and APK2 was absent except for the quiescent centre and APK1 and APK4 showed complementary patterns of expression. Remarkable is the strong expression of Apks, except Apk2, in the pollen. Interestingly, although seeds contain high levels of glucosinolates, the expression of Apks is very low and specific to the funiculus (Apk1 and -2).

The localisation of the Apks to the vasculature corresponds to that of several enzymes of the gls biosynthesis pathway which have been shown to be closely associated with vascular tissues in a number of studies (Reintanz et al., 2001; Schuster et al., 2006). A recent report has shown that the distribution of gls in Arabidopsis leaves is non-uniform (Shroff et al., 2008). Although gls are present throughout the leaf, the three major gls are more abundant in the tissues surrounding the main vein and the outer lamina (Shroff et al., 2008). This is believed to be due to a higher level being present at the parts of the leaf most vulnerable to damage by insects: the main vein needs to be protected from damage to prevent the disruption of the movement of nutrients and water through the leaf, and the edges of the leaf since these are most accessible to chewing insects. Myrosinase has been shown to be localised specifically in myrosin cells of the phloem parenchyma in Arabidopsis (Andréasson et al., 2001), and in guard cells (Andréasson et al., 2001; Thangstad et al., 2004). Koroleva et al. (2000) also demonstrated a high amount of gls in specific S-cells in flower stalks associated with vasculature. Localisation of PAPS synthesis at these sites would thus enable provision of the activated sulphate in situ without the need for long distance transport. Interestingly, such specific generation and location of other compounds active in plant stress responses in vascular bundles, e.g. jasmonates and their metabolites, were found also in other plant species (Stenzel et al., 2003; Schilmiller & Howe, 2005).

### 3. Apk1 and Apk2 are the Major APS Kinase Isoforms in Arabidopsis

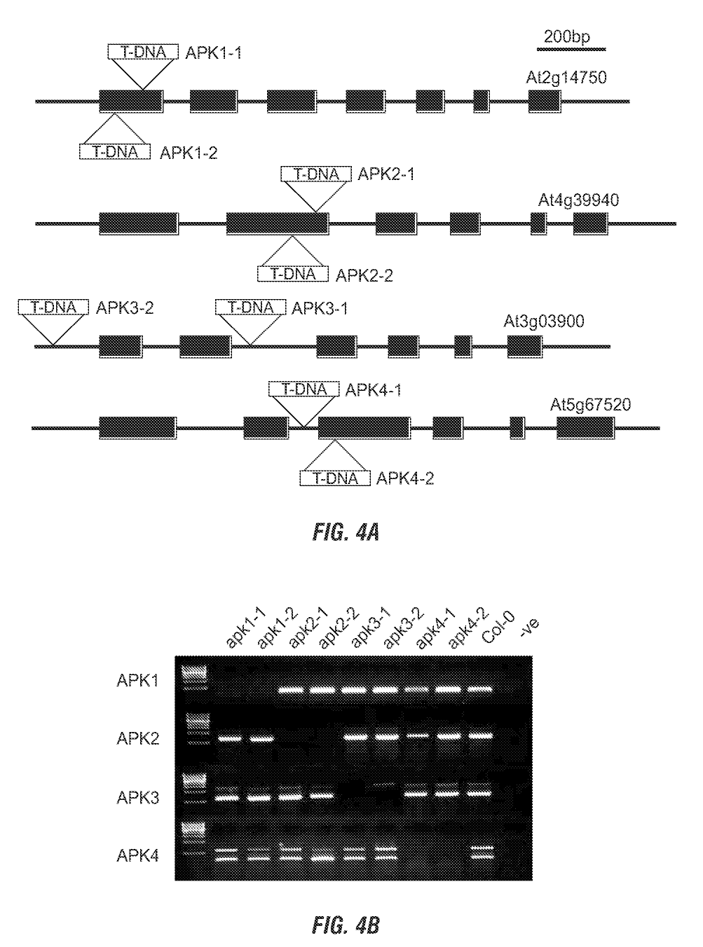

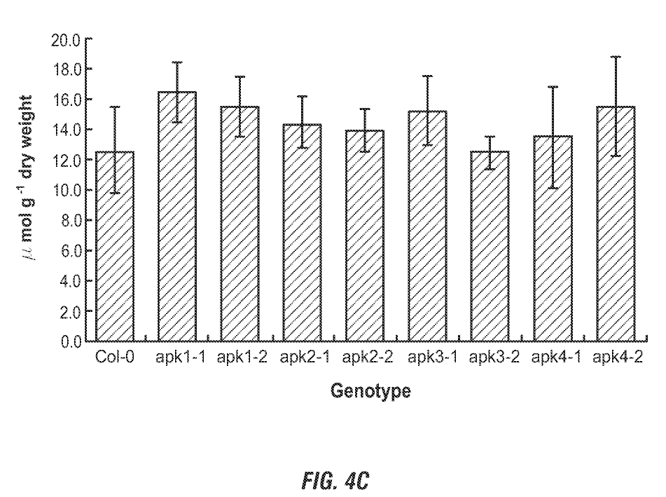

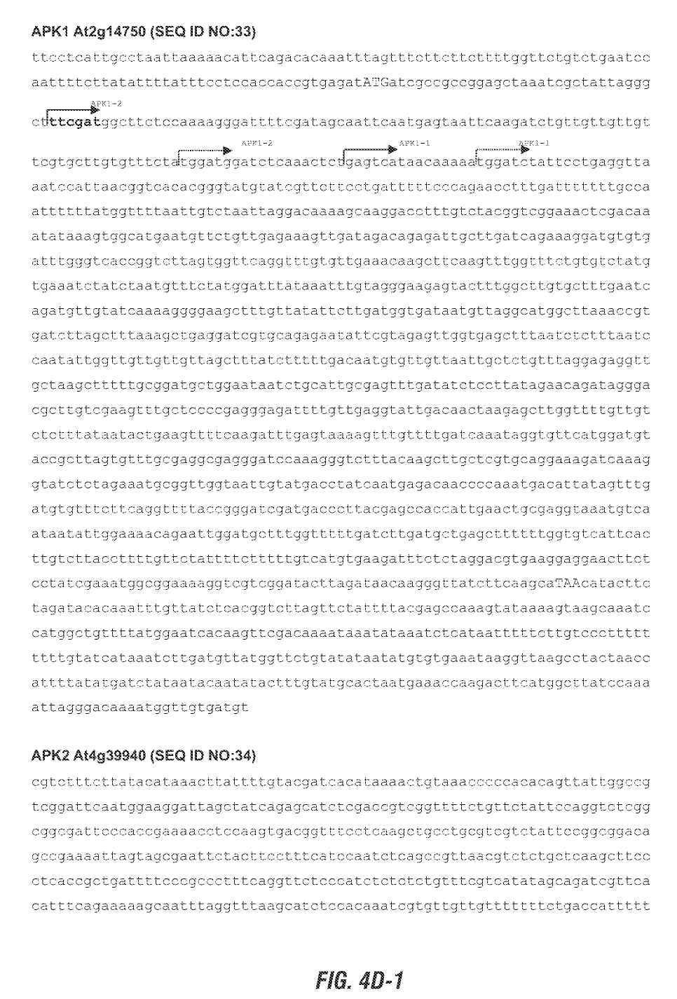

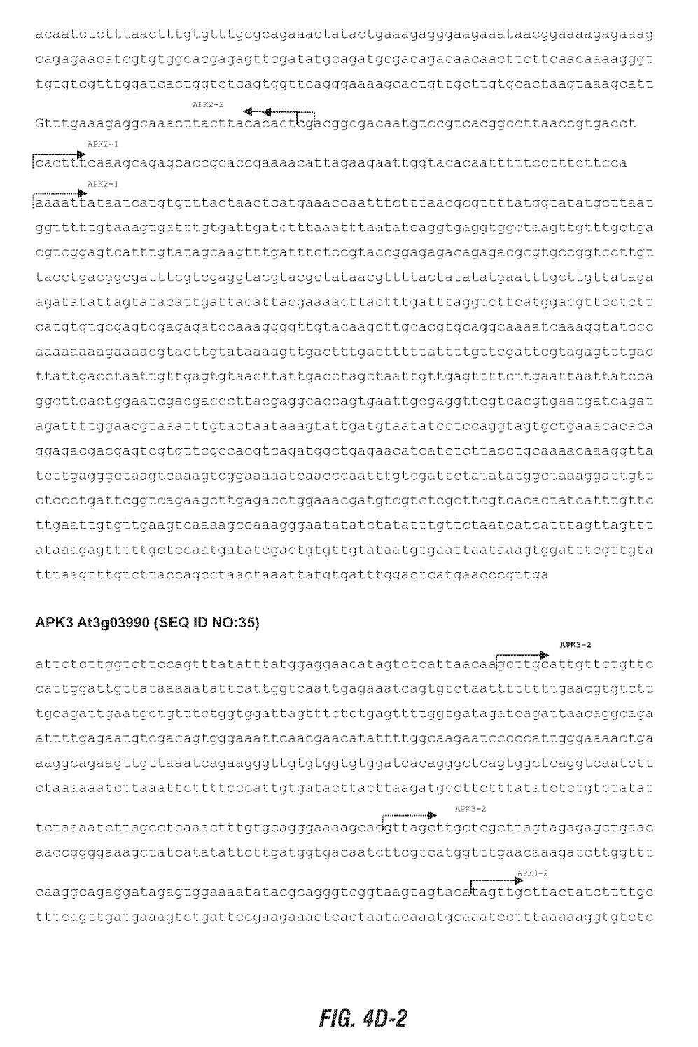

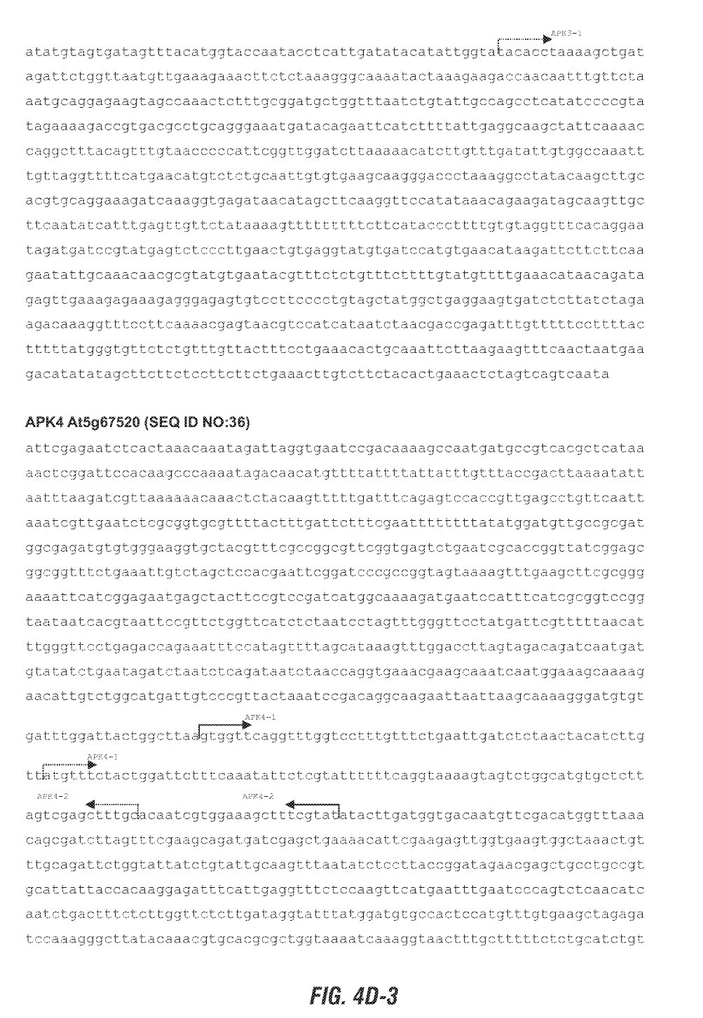

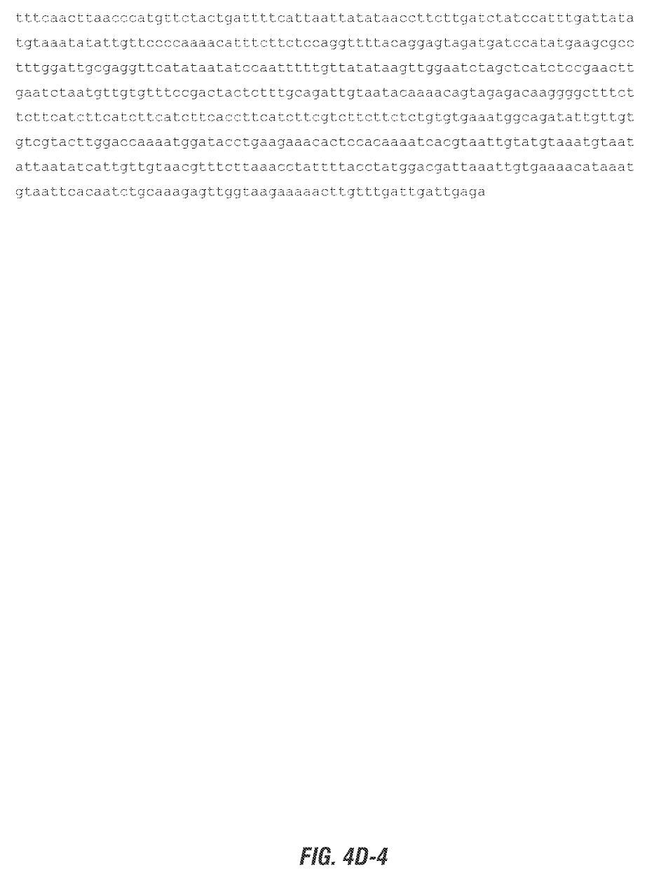

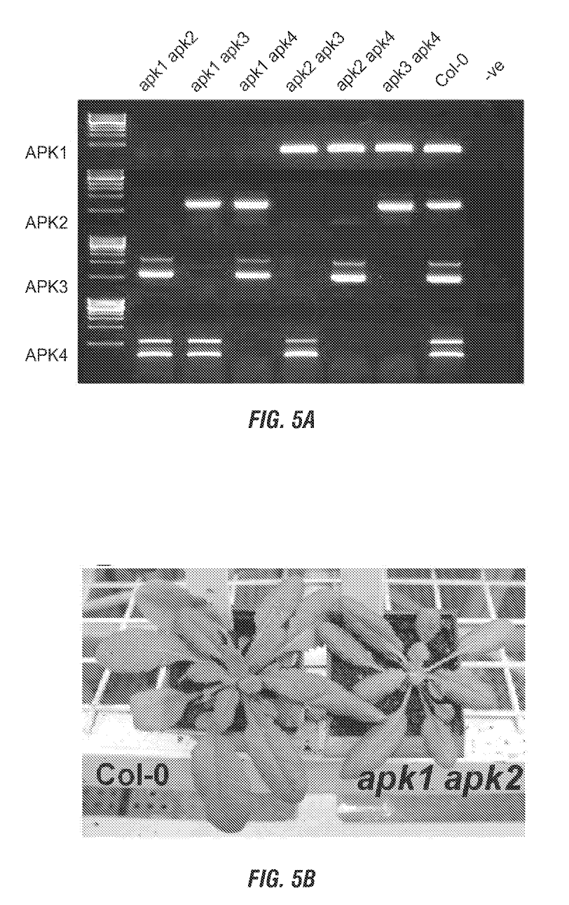

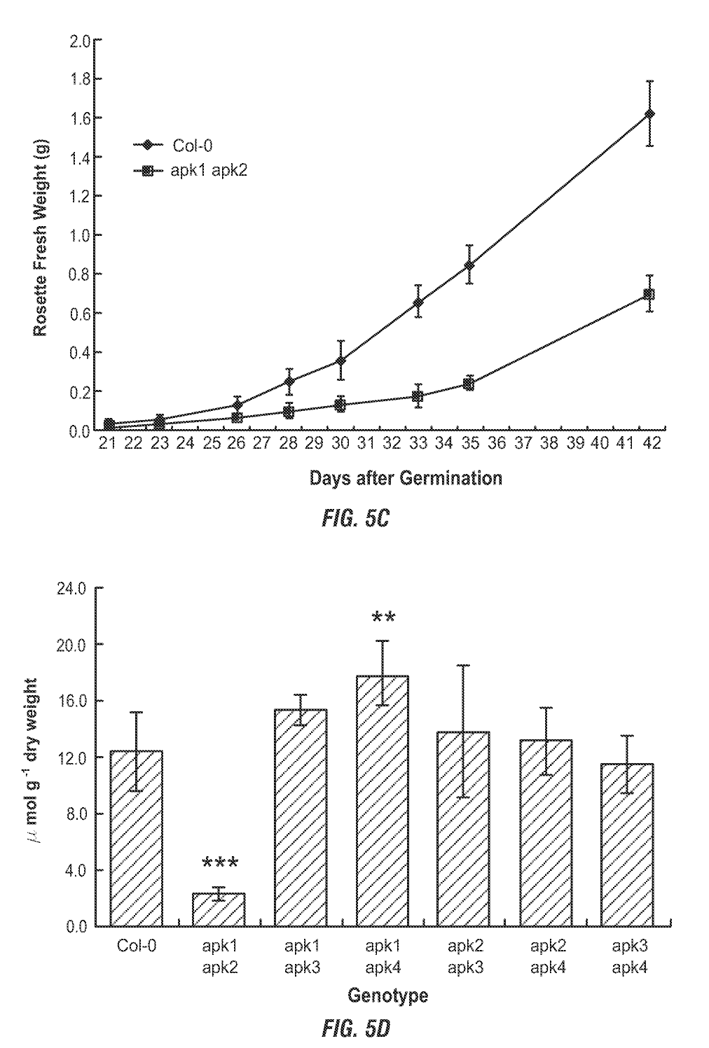

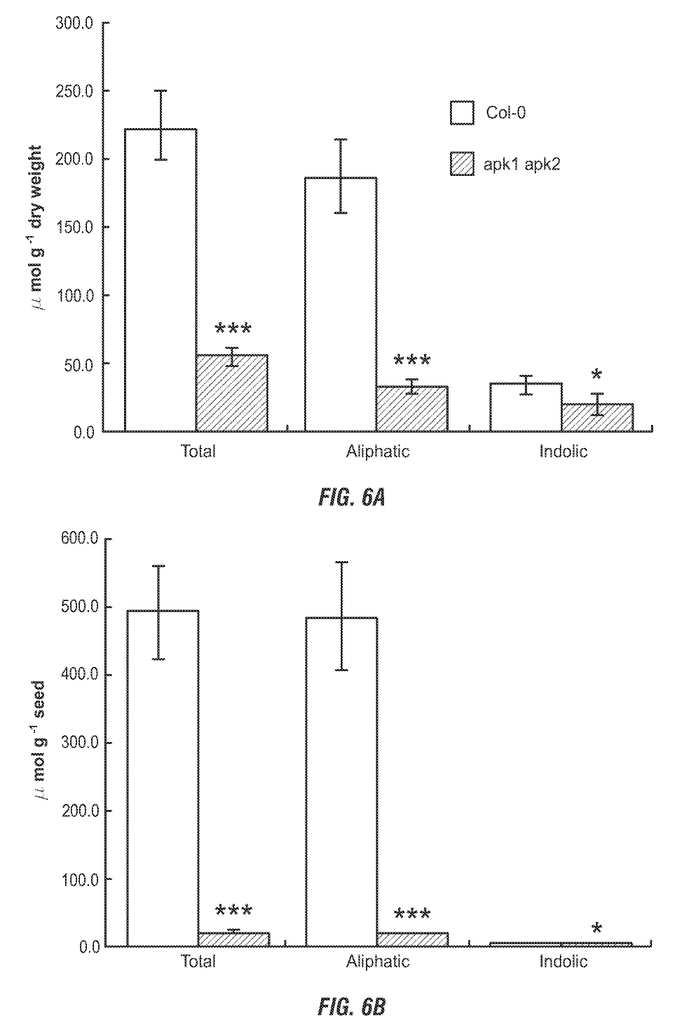

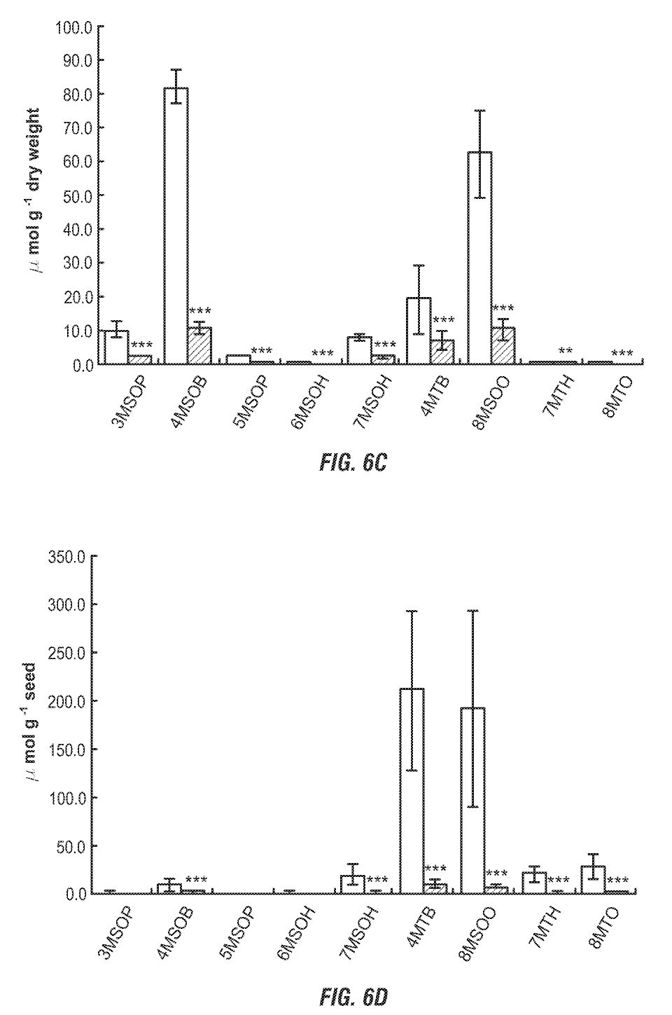

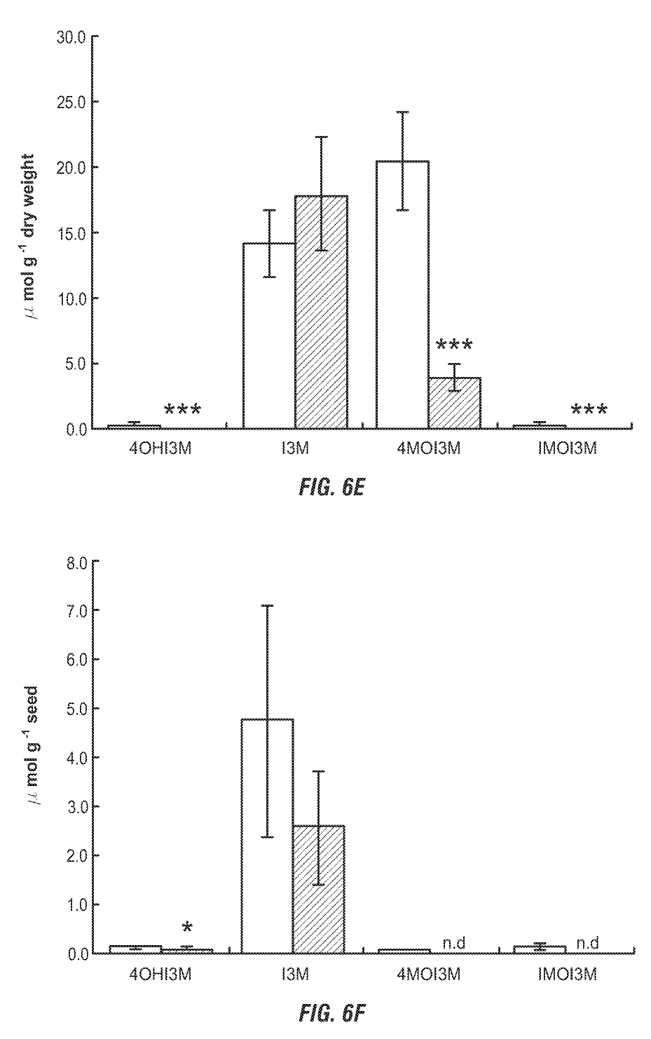

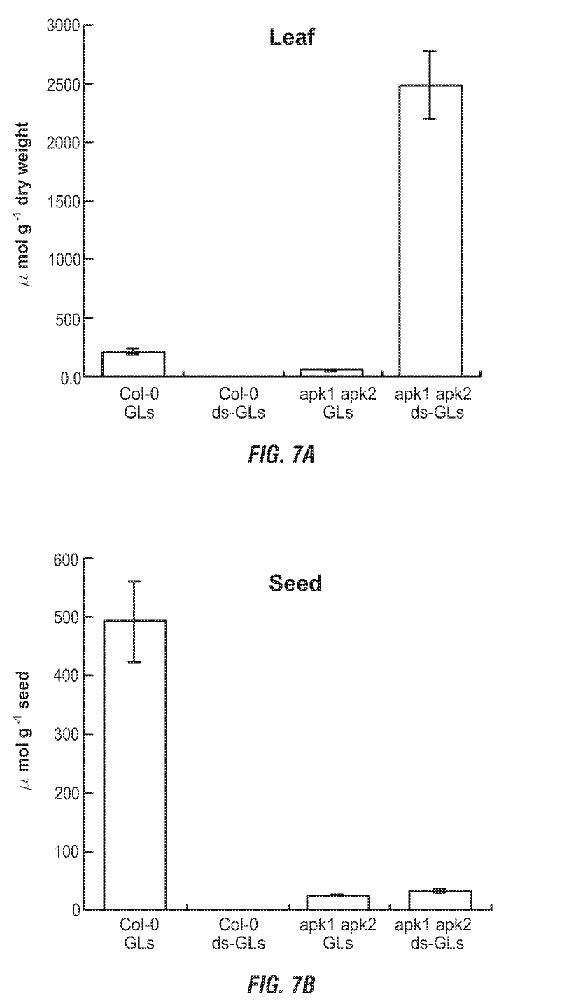

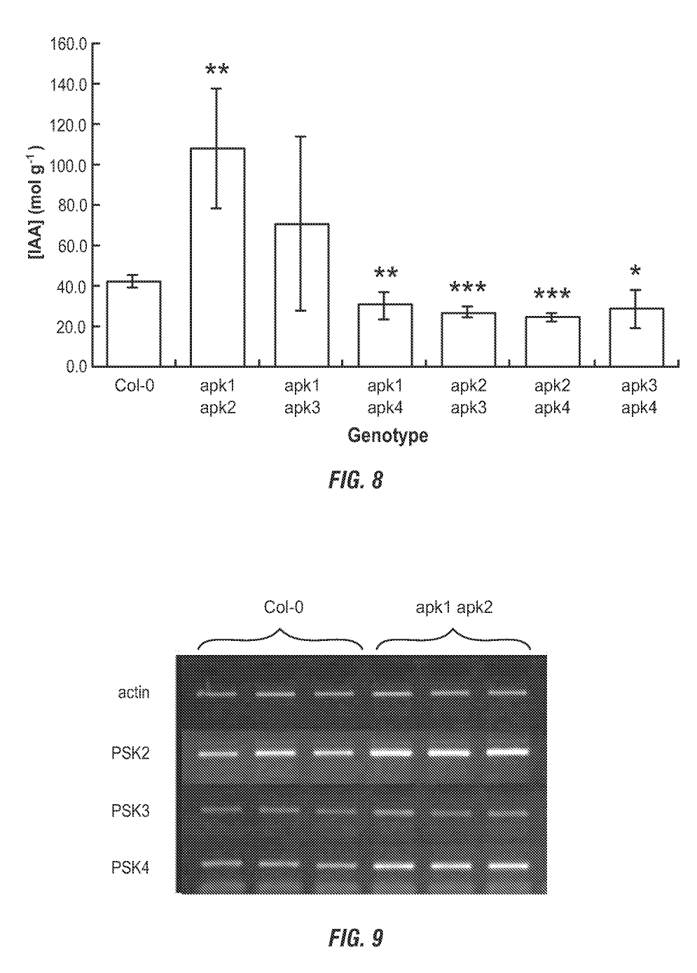

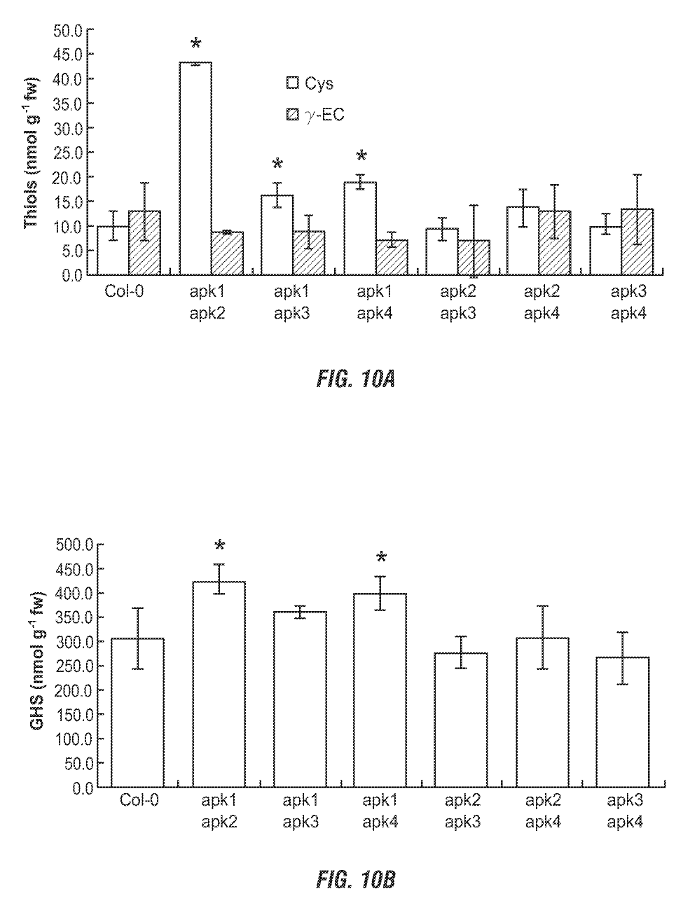

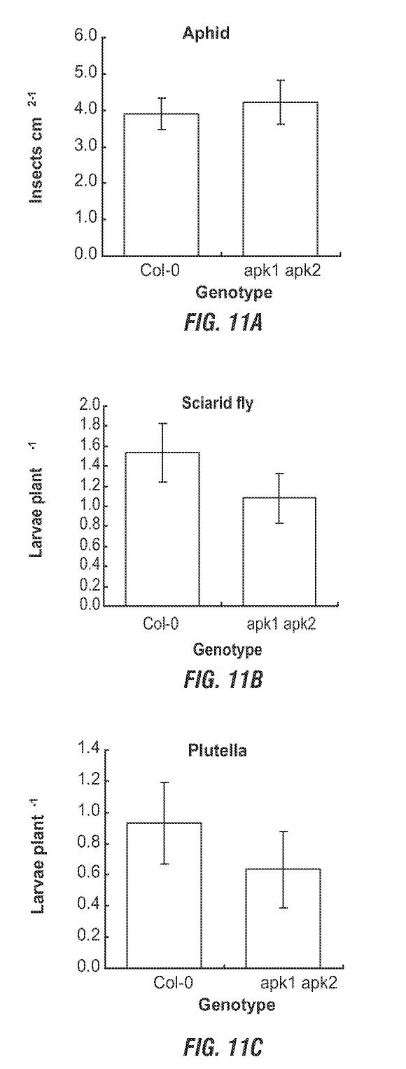

To determine specific functions of the APS kinase isoforms we analysed T-DNA insertion lines for all four genes (FIG. 4). The lack of an obvious phenotype in single mutants indicated that none of the Apk isoforms are essential for normal growth and seed set. This is presumably due to at least partial functional redundancy within the family. The double mutants, however, revealed that this redundancy is limited. The apk1 apk2 double mutant showed a clear growth phenotype (FIG. 5). The plants were smaller than wild-type plants, and showed delayed but normal flower and seed development. As gls are the major group of sulphated compounds in Arabidopsis we determined their content in all the mutant plants. Indeed, levels of total sulphated gls in the apk1 apk2 mutant were reduced 5-fold in mature leaves (FIG. 5D), and 14-fold in the seeds (FIG. 7). This indicates that Apk1 and Apk2 are the major Apk isoforms that provide PAPS for synthesis of gls and other sulphated metabolites. Apk1 and -2 on their own are able to provide sufficient PAPS for gls synthesis, as single knockout alleles of both isoforms contained normal or even slightly elevated levels of sulphated gls. Despite the reduction in leaf glucosinolates the apk1 apk2 plants were no more susceptible to insect pests than WT plants (FIG. 11).

These differences in double knock-out plants may be caused by specific functions of the individual isoforms or by an overall reduction in the APS kinase activity. Antibodies were raised against recombinant Apk1 to assess Apk protein accumulation in the mutant plants; however, the antiserum was specific to Apk1 and did not cross-react with other recombinant Apk isoforms. Also the transcripts for the individual isoforms are too diverse to allow a total Apk mRNA quantification.

### 4. Manipulation of APS Kinase Affects Glucosinolate Biosynthesis

Many Arabidopsis mutants have been characterised with low levels of total or specific gls (Bak et al., 2001; Reintanz et al., 2001 Grubb et al., 2004; Mikkelsen et al., 2004; Gigolashvili et al., 2007a, 2007b; Beekwilder et al., 2008). The underlying genes encoded enzymes of gls backbone biosynthesis or transcriptional factors and contributed significantly to the elucidation of the pathway and its regulation. We show here that manipulation of the last step in gls biosynthesis, the sulphation of ds-gls, also affects gls levels (FIG. 6). This figure shows a large reduction in glucosinolate in both the leaves (top panel) and the seeds (bottom panel) in the apk1 apk2 double mutant when compared to WT. It further shows that there is a greater change in the levels of aliphatic glucosinolates when compared to indolic glucosinolates in both leaves and seeds. The leaves maintain their protection against pests. The reduction seen in seeds (bottom panel) is greater than the reduction seen in leaves and leads to an increase in the palatability of the seeds when used as a feedstuff. Disruption of Apk1 and -2 leads to a reduced capacity to synthesize PAPS and thus the activated sulphate is less available. As the limiting step for gls synthesis in the apk1 apk2 mutant seems to be the provision of PAPS, we expected that the ds-gls precursors may accumulate. Indeed, in leaves of the apk1 apk2 mutant the ds-gls accumulated to very high levels. The content of ds-gls in Apk1 Apk2 mutant was 11-fold higher than that of intact gls in WT (FIG. 7). The ds-gls accumulation is much higher than would be expected from a simple reduction of concentration of the second substrate. This indicates that plants recognised the low level of glucosinolate products and increased the production of the desulphated precursors. This conclusion is supported by the coordinated upregulation of genes involved in gls biosynthesis. In addition, mRNA levels of several genes encoding transcription factors controlling gls biosynthesis were elevated in apk1 apk2 compared to WT. The increased levels of the biosynthetic genes thus may be caused by the induction of the MYB factors, which were expressed up to eight-fold higher than in WT (Table 2). Over expression of these transcription factors was shown to result in increased levels of mRNA for gls biosynthetic genes and accumulation of glucosinolates (Gigolashvili et al., 2007a, 2007b, 2008). Without wishing to be bound by mechanistic considerations, we can thus postulate a feedback regulation of gls biosynthesis by the end products. The build up of ds-gls in the leaves of the apk1 apk2 double mutant has substantially no effect on the degree of protection afforded by glucosinolates in these plants because the ds-gls precursors do not react with myrosinase and do not form the bioactive volatile reaction products, such as isothiocyanates or nitriles. Again, the reduction in levels of glucosinolates seen in leaves did not affect susceptibility of the apk1 apk2 plants to insects.

Surprisingly, in seeds of apk1 apk2 plants, the ds-gls did not accumulate, suggesting that intact gls are loaded into the developing seeds and not synthesised there, and the sulphate group is required for loading. This is in agreement with previous reports which concluded that gls and not ds-gls are the form which is transported in the phloem (Chen et al., 2001) and that fully-formed gls are loaded into the seeds via phloem from maternal tissue (Magrath & Mithen, 1993). In Sinapis alba, it has been shown that at least one gls destined for the seed tissue is synthesised in the silique wall and transported into the seed (Du & Halkier, 1998). The very specific expression of Apk1 and Apk2 in the funiculus in Arabidopsis may indicate that ds-gls are synthesised in siliques and sulphated in the funiculus during transport into the developing seeds.

The analysis of individual gls and ds-gls revealed that the desulpho-precursors of I3M accumulated in leaves, but the ds-gls of the modified indolic gls were undetectable. This corroborates the hypothesis that the modifications of the indolic gls occur after sulphation to form I3M (Kim & Jander, 2007), and contradicts the findings of Pedras and Montaut (2004), who hypothesised that the aldoxime moiety is modified early in biosynthesis (Pedras & Montaut, 2004).

The fact that I3M was sulphated in apk1 apk2 to higher degree than the aliphatic gls may be explained by different PAPS pools provided by different Apk isoforms for indolic and aliphatic ds-gls sulphation. It has been shown previously that upon attack by the insect Myzus persicae, I3M is converted rapidly to 4MOI3M, as this is less stable than I3M and more likely to break down spontaneously in the insect gut, as this pest inserts its stylet intracellularly, thus avoiding the myrosinase defence system (Kim & Jander, 2007). It is thus plausible to hypothesise that a hierarchy of individual gls exists with the I3M being synthesised preferentially. When PAPS supply is not limiting all gls are made, if, however it is limiting, such as in the apk1 apk2 mutant, the PAPS is preferentially used to synthesise I3M at the expense of other gls. This would provide the plant even at a lower total gls levels with the most effective protection against a broad variety of bacterial diseases (Clay et al., 2009) in antifungal defence (Bendarek et al., 2009) as well as against herbivores.

TABLE 2
Fold-changes of transcript levels of genes involved in glucosinolate
biosynthesis and its transcriptional control in apk1 apk2 plants.

| Gene | AGI | Description | Fold-change | p |
|---|---|---|---|---|
| Apk1 | At2g14750 | APS kinase 1 | 0.0111 | 0.00000002 |
| Apk2 | At4g39970 | APS kinase 2 | 0.0294 | 0.00493 |
| Apk3 | At3g03900 | APS kinase 3 | 1.070 | 0.568 |
| Apk4 | At5g67520 | APS kinase 4 | 0.548 | 0.233 |
| | At3g19710 | Methionine-oxo acid transaminase | n/r | — |
| MAM1 | At5g23010 | Methylthioalkylmalate synthase 1 | 3.13 | 0.000045 |
| MAM3 | At5g23020 | Methylthioalkylmalate synthase 3 | 8.318 | 0.0000073 |
| ASA1 | At5g05730 | Anthranilate Synthase Alpha subunit | 1.98 | 0.0170 |
| ASB1 | At1g25220 | Anthranilate Synthase Beta subunit | n/r | — |
| TSA1 | At4g02610 | Tryptophan synthase alpha subunit 1 | 1.24 | 0.0157 |
| TSB1 | At5g54810 | Tryptophan synthase beta subunit 1 | n/r | — |
| CYP79A2 | At5g05260 | CYTOCHROME P450 79A2 | n/e | — |
| CYP79F1/SUS1 | At1g16410 | CYTOCHROME P450 79F1, SUPERSHOOT1 | n/r | — |
| CYP79F2 | At1g16400 | CYTOCHROME P450 79F2 | n/r | — |
| CYP79B2 | At4g39950 | CYTOCHROME P450 79B2 | 8.524 | 0.000125 |
| CYP79B3 | At2g22330 | CYTOCHROME P450 79B3 | 4.967 | 0.000268 |
| CYP83A1 | At4g13770 | CYTOCHROME P450 83A1 | 2.313 | 0.000389 |
| CYP83B1/SUR2 | At4g31500 | CYTOCHROME P450 83B1, SUPERROOT2 | 1.918 | 0.000362 |
| SUR1 | At2g20610 | Transaminase, SUPERROOT1 | 2.076 | 0.000212 |
| UGT74B1 | At1g24100 | UDP-Glucosyltransferase 74B1 | 2.389 | 0.00246 |
| UGT74C1 | At2g31790 | UDP-Glucosyltransferase 74C1 | 2.225 | 0.0000785 |
| SOT16 | At1g74100 | Sulphotransferase, indolic glucosinolates | 2.097 | 0.00489 |
| SOT17 | At1g18590 | Sulphotransferase, aliphatic glucosinolates | 4.186 | 0.000146 |
| SOT18 | At1g74090 | Sulphotransferase, aliphatic glucosinolates | 2.291 | 0.000278 |
| | At1g65880 | Benzoate-CoA ligase | n/e | — |
| AOP1 | At4g03070 | 2-oxoglutarate-dependant dioxygenase | 1.09 | 0.476 |
| AOP2 | At4g03060 | 2-oxoglutarate-dependant dioxygenase | 3.15 | 0.000150 |
| AOP3 | At4g03050 | 2-oxoglutarate-dependant dioxygenase | n/e | — |
| HAG1/MYB28 | At5g61420 | High Aliphatic Glucosinolate 1 R2R3-MYB transcription factor | 1.326 | 0.0185 |
| HAG2/MYB76 | At5g07700 | High Aliphatic Glucosinolate 2 R2R3-MYB transcription factor | 7.694 | 0.0000208 |
| HAG3/MYB29 | At5g07690 | High Aliphatic Glucosinolate 3 R2R3-MYB transcription factor | 2.370 | 0.0277 |
| HIG1/MYB51 | At1g18570 | High Indolic Glucosinolate 1 R2R3-MYB transcription factor | 1.325 | 0.060 |
| HIG2/MYB122 | At1g74080 | High Indolic Glucosinolate 2 R2R3-MYB transcription factor | n/e | — |
| HIG3/MYB34/ATR1 | At5g60890 | High Indolic Glucosinolate 3 R2R3-MYB transcription factor | 15.796 | 0.116 |
| OBP2 | At1g07640 | DOF transcription factor | 0.870 | 0.222 |
| IQD1 | At3g09710 | Calmodulin binding protein | 0.610 | 0.374 |
| FRY1/SAL1 | At5g63980 | Fiery1/Sal1, 3′(2′),5′-bisphosphate nucleotidase activity. Detox | 2.044 | 0.000453 |
| | | of PAP | | |

n/e = not expressed
n/r = not represented on Affymetrix chip.
Transcripts highlighted in bold are significantly altered in the Apk1 Apk2 mutant compared to Col-0 samples at p < 0.05.

### 6. Disruption of Apk1 and Apk2 Affects Other Sulphated Compounds

As all four Apk isoforms are functional and disruption of Apk1 and -2 results in reduction of gls the inventors investigated whether levels of other sulphated compounds are also altered. As not much is known about sulphated compounds in Arabidopsis apart from gls the inventors turned to the only sulphated metabolite that can be reliably determined, the 12-HSO₄-JA (Miersch et al., 2008). In apk1 apk2 mutant the level of 12-HSO₄-JA was reduced to the same extent as the levels of gls, indicating that the SOTs synthesising this compound use the same PAPS pool (Table 3). The levels of 11-OH-JA and 12-OH-JA which is the precursor of 12-HSO₄-JA, were elevated in these plants but not to such high levels as ds-gls. This is another indication that the synthesis of ds-gls is indeed actively upregulated in the apk1 apk2 plants in contrast to simple accumulation of unused substrates. On the other hand, in apk1 apk2 mutant plants 12-O-Glc-JA was increased showing that when the 12-OH-JA cannot be sulphated, alternative routes of its metabolism are enhanced.

However, in contrast to gls, we observed significant alterations in 12-HSO₄-JA levels also by other mutants in Apk. In all genotypes lacking APK1 this compound was significantly reduced, which was mostly accompanied by increase of 12-OH-JA (Table 3). These results indicate that either APK1 is specifically associated with the SOT involved in jasmonate sulphation or that already in single apk1 mutants the PAPS synthesis is compromised so that it is not sufficient for all sulphated compounds. Glucosinolates as major metabolites in defence against biotic stress might thus be synthesised as a priority and the other compounds, such as 12-HSO₄-JA only when enough PAPS is available. This may not be the case in the apk1 -1, apk1 apk3, and apk1 apk4 plants, where the 12-HSO₄-JA is reduced.

TABLE 3
Levels of jasmonate and its derivatives in Apk mutants

| | | | | | | 12-O-Glc- |
|---|---|---|---|---|---|---|
| Genotype | OPDA^{a} | JA | 11-OH-JA | 12-OH-JA | 12-HSO₄-JA | JA |
| Col-0 | 4451.3 ± 1072.8 | 86.8 ± 65.5 | 145.8 ± 62.9 | 180.0 ± 18.8 | 192.3 ± 31.5 | 89.8 ± 104.9 |
| apk1-1 | 5225.3 ± 680.9 | 51.8 ± 35.3 | 146.8 ± 11.0 | 246.8 ± 59.3 | 149.8 ± 24.9 | 106.5 ± 88.1 |
| apk2-1 | 6286.8 ± 658.2* | 105.0 ± 32.4 | 161.8 ± 11.8 | 163.8 ± 24.8 | 172.5 ± 30.6 | 87.8 ± 65.9 |
| apk1 | 2639.3 ± 583.4* | 84.8 ± 32.1 | 254.8 ± 64.3* | 235.0 ± 13.4* | 42.0 ± 12.1* | 198.5 ± 27.0* |
| apk2 | | | | | | |
| apk1 | 5508.0 ± 359.2 | 76.8 ± 48.6 | 127.3 ± 47.3 | 199.3 ± 28.1 | 133.8 ± 31.4* | 77.0 ± 98.2 |
| apk3 | | | | | | |
| apk1 | 7684.5 ± 1199.4* | 94.5 ± 54.2 | 103.3 ± 20.3 | 219.8 ± 74.5 | 146.5 ± 41.5* | 64.5 ± 74.6 |
| apk4 | | | | | | |
| apk2 | 4929.8 ± 1196.8 | 163.8 ± 123.2 | 171.3 ± 24.6 | 298.3 ± 67.7* | 179.8 ± 81.1 | 56.8 ± 113.5 |
| apk3 | | | | | | |
| apk2 | 5482.8 ± 783.4 | 117.8 ± 52.2 | 196.3 ± 38.0 | 312.8 ± 79.5* | 295.3 ± 34.6* | 110.0 ± 74.6 |
| apk4 | | | | | | |
| apk3 | 5449.0 ± 361.2 | 123.0 ± 52.3 | 148.5 ± 32.2 | 239.0 ± 31.3* | 689.5 ± 400.0* | 42.5 ± 60.1 |
| apk4 | | | | | | |

^{a}measurements are expressed as pmol g⁻¹ fresh tissue and are the mean +/− SD of four replicates.
Asterisks mark values significantly different from wild type at P < 0.05.

### 7. Interconnection Between Glucosinolate Synthesis and Hormone Homeostasis

Many mutants in gls biosynthesis are also affected in auxin homeostasis (Delarue et al., 1998; Barlier et al., 2000; Bak et al., 2001; Reintanz et al., 2001; Grubb et al., 2004; Mikkelsen et al., 2004). This is because indole-3-acetaldoxime, the precursor of indolic gls is also an intermediate in the biosynthesis of IAA (Mikkelsen et al., 2004). For example, the sur1 and sur2 plants, deficient in C-S lyase and cytochrome P-450 CYP83B1, respectively, have severely reduced indolic gls levels and increased IAA content, therefore displaying a superroot phenotype (Barlier et al., 2000; Mikkelsen et al., 2004). The same is true for another mutant, bus1-1 (bushy), disrupted in CYP79F1, which also accumulates auxin, while the aliphatic gls are severely reduced (Reintanz et al., 2001). The apk1 apk2 plants also possessed significantly lowered gls levels, however, they did not display any of the typical high auxin phenotypes: elongated hypocotyl, epinastic cotyledons, crinkled leaves, increased number of lateral roots, multiple adventitious roots, enhanced number of lateral shoots, altered flower morphology or (semi)sterility. Therefore, we did not expect any alterations in auxin levels in these plants. Nevertheless, in 5-week-old rosette leaves of apk1 apk2, but no other double mutant, the levels of IAA were 3-fold higher than in wild type leaves. In addition, the microarray analysis revealed that expression of several genes involved in IAA synthesis was increased in apk1 apk2 leaves compared to wild-type. This clearly shows that even though the gls level was modified by a completely different route, the effect on auxin still remained. The lack of high auxin phenotype, which was observed also in plants accumulating IAA due to over expression of ATR1/MYB34 transcription regulator (Celenza et al., 2005), can be explained by different extent of IAA accumulation, developmental stage when IAA accumulates, or variation in levels of IAA conjugates found in sur2 plants.

The apk1 apk2 plants, however, displayed a clear morphological phenotype, although different from the typical high auxin one. The plants were smaller throughout the whole vegetative growth phase and set flower later than WT plants. Reduction in aliphatic glucosinolates and both indolic and aliphatic gls in plants with reduced expression of HAG1/MYB28 and HIG1/MYB51 transcription factors had no effect of plant growth (Gigolashvili et al., 2007a, 2007b), therefore, gls are unlikely to be responsible for this growth phenotype. The key thus might be another sulphated metabolite. As beside gls also the 12-HSO₄-JA content was reduced in apk1 apk2 plants we might reasonably expect that also the level of other sulphated molecules would be decreased and some might be responsible for the slower growth. The decrease in 12-HSO₄-JA itself is unlikely to cause the growth retardation as this metabolite was reduced also in other double mutants that grew normally. Possible candidates are thus the sulphated peptides, PSK and PSY1, which have been associated with regulation of growth (Matsubayashi & Sakagami 1996; Amano et al., 2007). Disruption of receptors for PSK and PSY1 leads to premature senescence of rosette leaves, gradual loss of the ability to form calluses and to a semi-dwarf phenotype (Matsubayashi et al., 2006; Amano et al., 2007). Although we have not observed premature senescence in apk1 apk2 plants their smaller size resembles the effect of the lost ability to perceive PSY1 signals. We have not measured PSK or PSY1; however, this hypothesis is strongly supported by the observed increase in mRNA levels for PSK2 and PSK4 precursors in apk1 apk2 plants. As the decrease in gls is accompanied by increase in ds-gls and lower levels of 12-HSO₄-JA by higher 12-OH-JA, it is highly probable that the increase in PSK precursors is linked to the decrease of the mature PSKs. The molecular basis of the growth phenotype of apk1 apk2 plants as well as general effects of the reduction in APS kinase on plant metabolism.

The results in relation to PSKs are interesting, since the apk1 apk2 double mutants are semi-dwarf. This suggests that sulphated compound(s) is/are required for normal growth. These compounds are probably not glucosinolates, since mutants in gls synthesis do not show this phenotype. It is suggested that sulphated peptides are responsible for this semi-dwarf phenotype.

### 8. Effects of Apk Disruption on Primary Metabolism

The disruption of APK1 and APK2 resulted in a decrease in synthesis of sulphated compounds with further consequences for sulphur metabolism. The obvious expectation was that diminishing sulphur flux into PAPS would enable greater flux through the primary sulphate reduction. Indeed, steady state levels of cysteine and glutathione were higher in leaves of apk1 apk2 plants than in WT leaves. However, the alterations in sulphur metabolism were far more complicated than this simple effect and included potential accumulation of upstream compounds as well as feedback signals from downstream metabolites. Despite the reduction in rate of its metabolization, APS content diminished in the apk1 apk2 plants. This was probably caused by the increase in APS reduction rate as demonstrated by the increase in thiol content. However, this was not seen on the level of APR activity, which was not affected in any of the Apk double mutants. The increased thiol content was surprisingly accompanied by an increase in sulphate accumulation. Several genes encoding sulphate transporters and two isoforms of ATPS were induced in the apk1 apk2 plants, presumably in an attempt to increase the provision of PAPS. This agrees with a recent report showing an upregulation of sulphate assimilation due to over expression of the MYB transcription factors HAG3/MYB29, HAG2/MYB76, HIG1/MYB51, and HIG3/ATR1/MYB34 (Malitsky et al. 2008). The increased expression of the sulphate transporter genes might be responsible for the increased sulphate content despite the higher synthesis of thiols. The increase in O-acetylserine in apk1 apk2 is intriguing, as this compound is considered to be involved in signaling of sulphur status of the plant (Koprivova et al., 2000, Hirai et al., 2003); however, the alterations of the pathway in apk1 apk2 plants are not consistent with the described effects of O-acetylserine on various components of primary sulphate assimilation. The analysis of the apk1 apk2 plants thus revealed unexpectedly strong and complex interconnection between gls accumulation and primary sulphate metabolism.

Having generally described this invention, the following examples are provided to further describe and enable the various embodiments of this invention, as reflected in the appended claims. Those skilled in the art will appreciate that the details provided in the following examples are not intended as limiting, but rather to fully enable and describe this invention, including its best mode. For an understanding of the scope of the invention disclosed and claimed herein, those skilled in the art are referred to the appended claims and the equivalents thereof.

### EXAMPLES

### Methods

### Plant Materials and Growth Conditions

Arabidopsis thaliana (ecotype Columbia) plants were used as WT in this study. Unless otherwise stated, all plants were grown in a controlled environment chamber under a short day 10-h-light/14-h-dark cycle at constant temperature of 22° C., 60% relative humidity, and light intensity of 160 μmol m⁻² s⁻¹. A minimum of four whole rosettes were harvested at 5 weeks old and immediately frozen in liquid nitrogen. Tissue was then either freeze-dried (for gls and ds-gls analysis) or ground under liquid nitrogen in a pestle and mortar (for RNA extraction, jasmonate and IAA measurements). Plants for seeds were grown in a standard glasshouse. To compare root phenotypes of the apk1 apk2 mutants and WT plants the seeds were plated on 0.8% agarose plates containing Murashige & Skoog medium supplemented with 3% sucrose and after 4 days at 4° C. in the dark grown vertically in the controlled environment chamber.

### Screening of T-DNA Insertion Mutants

Putative T-DNA insertion mutants of Arabidopsis were obtained from Nottingham Arabidopsis Resource Centre (NASC). All T-DNA lines were in the Columbia background. Two independent alleles were obtained for each isoform namely SALK_{—}053427 (apk1-1); SALK_{—}034586 (apk1-2); SALK_{—}093072 (apk2-1); SALK_{—}077590 (apk2-2); SALK_{—}115182 (apk3-1); SALK_{—}145507 (apk3-1); SALK_{—}035815 (apk4-1); and SALK_{—}068062 (apk4-2). Homozygous mutants were identified using primer LBb1 and an appropriate gene-specific primer. Primer sequences for the identification of homozygous T-DNA insertions are listed in Table 4.

TABLE 4

Primer sequences for identification of T-DNA
insertions in Apk mutants

| | | | |
|---|---|---|---|
| Mutant | Mutant ID | 5′ Primer | 3′ Primer |

| | | | |
|---|---|---|---|
| apk1-1 | SALK_0534 | CCACAACTGCATATAGACTATGC | GACCCAAATCACACATCC |
| | 27 | (SEQ ID NO:5) | (SEQ ID NO:6) |
| apk1-2 | SALK_0345 | CCACAACTGCATATAGACTATGC | GACCCAAATCACACATCC |
| | 86 | (SEQ ID NO:5) | (SEQ ID NO:6) |
| apk2-1 | SALK_0930 | TAACGTCTCTGCTCAAGC | ATGTTTTCGGTGAGGTGC |
| | 72 | (SEQ ID NO:7) | (SEQ ID NO:8) |
| apk2-2 | SALK_0775 | TAACGTCTCTGCTCAAGC | ATGTTTTCGGTGAGGTGC |
| | 90 | (SEQ ID NO:7) | (SEQ ID NO:8) |
| apk3-1 | SALK_1151 | GCGCATATGTCGACAGTGGGAAATTC | CTATCACTACATATGAGACACC |
| | 82 | (SEQ ID NO:9) | (SEQ ID NO:10) |
| apk3-2 | SALK_1155 | CCGTGATTATCTCTGTCAGC | TTCCACTCTATCCTCTGC |
| | 07 | (SEQ ID NO:11) | (SEQ ID NO:12) |
| apk4-1 | SALK_0358 | ACTTCCGTCCGATCATGG | GCTCGATCATCTGCTTCG |
| | 15 | (SEQ ID NO:13) | (SEQ ID NO:14) |
| apk4-2 | SALK_0680 | ACTTCCGTCCGATCATGG | GCTCGATCATCTGCTTCG |
| | 72 | (SEQ ID NO:13) | (SEQ ID NO:14) |
| LBb1 | all | GCGTGGACCGCTTGCTGCAACTC | |
| | | (SEQ ID NO:15) | |

### DNA Extraction and Amplification for Screening T-DNA Insertion Lines

Crude DNA preparations were prepared by homogenising ca. 50 mg young rosette leaves in a 1.5 ml eppendorf tube in 400 pl extraction buffer (200 mM Tris-HCl pH 7.5; 250 mM NaCl; 25 mM EDTA; 0.5% SDS). Samples were vortexed and spun at 13,000 rpm for 5 min. 300 μl supernatant was transferred to a fresh tube containing 300 μl isopropanol. Samples were mixed and spun at 13,000 rpm for 5 min. Pellets were washed in 70% ethanol, dried, and resuspended in 100 μl SDW. 2.5 μl crude extract was used for amplification in 25 μl reaction by GoTaq Flexi DNA Polymerase (Promega).

### RNA Extraction and Expression Analysis

To verify absence of Apk transcripts in the T-DNA lines total RNA was isolated from leaves by phenol:chloroform:isoamylalcohol (25:24:1) extraction and LiCl precipitation. Aliquots of 500 ng cDNA were reverse transcribed using SuperScriptII Reverse Transcriptase (Invitrogen), according to the manufacturer's instructions. PCR was done with GoTaqFlexi DNA Polymerase in 25 μl reaction volume with primers specific to individual isoforms and 35 cycles (Table 5). The quantitative RT-PCR to verify the microarray results was performed exactly as described in Gigolashvili et al., (2007a).

TABLE 5

Primer sequences for RT-PCR analysis of Apk
transcripts in T-DNA lines

| | | |
|---|---|---|
| Gene | 5′ Primer | 3′ Primer |

| | | |
|---|---|---|
| APK_1 | GCTTTCGATGGCTTCTTC | GACCCAAATCACACATCC |
| | (SEQ ID NO:16) | (SEQ ID NO:6) |
| APK_2 | TAACGTCTCTGCTCAAGC | ATGTTTTCGGTGAGGTGC |
| | (SEQ ID NO:7) | (SEQ ID NO:8) |
| APK_3 | AACTGAAAGGCAGAAGTTG | TTCCACTCTATCCTCTGC |
| | (SEQ ID NO:17) | (SEQ ID NO:12) |
| APK_4 | ACTTCCGTCCGATCATGG | GCTCGATCATCTGCTTCG |
| | (SEQ ID NO:13) | (SEQ ID NO:14) |

### Microarray Analysis

The RNA was extracted from 5-week old rosette leaves by phenol:chloroform:isoamylalcohol (25:24:1) extraction and LiCl precipitation, treated with DNAseI and repurified using the Qiagen RNeasy Plant Mini Kit according to the manufacturer's instruction. The labelling, hybridization, and detection using 3 biological replicates of both WT and apk1 apk2 plants and ATH1 chip was performed by the NASC's International Affymetrix Service. Only genes flagged by the scanner software as “present” in all six samples were included in further analysis. The expression data were normalised according to the AtGenExpress recommendations using a global mean normalisation excluding the top and bottom 2% of the data. Fold-changes in expression levels were calculated from means of the three biological replicates and their statistical significance was tested by a T-test on the Log-transformed signal intensities. In addition, a false discovery rate control was performed according to Storey & Tibshinari, (2003), setting the threshold q value at 0.05. Iterative group analysis was employed to identify categories of genes which are over-represented among the genes differing significantly in expression levels between the two genotypes (Breitling et al., 2004). The microarray data are accessible through the NASCArrays service (http://affymetrix.arabidopsis.info/narrays/experimentbrowse.pl) under accession number NASCARRAYS-457.

Heterologous Expression of Recombinant Apk in E. coli

Coding regions of Apk isoforms were amplified from cDNA without the putative target peptides and cloned into pET 14b expression vector (Novagen). Primer sequences can be found in Table 6. Constructs were transformed into BL21(DE3) cells (Novagen), 50 ml cultures grown overnight at 28° C. in LB. Cells were disrupted by sonication and proteins purified using the His-Bind Purification Kit (Novagen) according to the manufacturers instructions. Purified protein was quantified and used in activity assays.

TABLE 6

Primer sequences for the pET14b constructs for
heterologous expression of recombinant Apks

| | Cloning | | |
|---|---|---|---|
| Gene | site | 5′-Primer^{a} | 3′-Primer |
| APK1 | NdeI/ | GCGCATATGGTTTCTATGGATGGATCTC | CGCTCGAGTTATGCTTGAAGATAACC |
| | XhoI | (SEQ ID NO:18) | (SEQ ID NO:29) |
| APK2 | NdeI/ | GCGCATATGGCCGTTAACGTCTCTGCTC | GCGGATCCTTAGCCCTCAAGATAACC |
| | BamHI | (SEQ ID NO:20) | (SEQ ID NO:21) |
| APK3 | NdeI/ | GCGCATATGTCGACAGTGGGAAATTC | GCGGATCCTTACTCGTTTTGAAGGAAACC |
| | BamHI | (SEQ ID NO:9) | (SEQ ID NO:22) |
| APK4 | XhoI/ | GCGCTCGAGTCCGGTGAAACGAAGCAAATC | GCGGATCCTTACATACAATTACGTGATTTTG |
| | BamHI | (SEQ ID NO:23) | (SEQ ID NO:24) |

^{a}Underlined sequences correspond to the indicated restriction sites used for cloning into the pET14b vector

### Enzyme Assays

Apk activity was measured using the coupled spectrophotometric assay of Renosto et al., 1984. The assay was however not sensitive enough to analyse plant extracts. APR activity was determined as the production of [³⁵S]sulphite, assayed as acid volatile radioactivity formed in the presence of [³⁵S]APS and dithioerythritol as reductant (Koprivova et al., 2000). ATPS was measured as the APS and pyrophosphate-dependent formation of ATP (Cumming et al., 2007). The protein concentrations were determined according to Bradford with bovine serum albumin as a standard.

### Creation of Transgenic Plants Expressing APKₚᵣₒ:GUS

3.5 kb fragments of genomic DNA immediately upstream of the ATG codons of each Apk isoform were amplified using the EasyA polymerase (Stratagene). The primers were designed to add attB1 and attB2 sites to the fragments, which were cloned into the TOPO XL cloning vector (Invitrogen). The fragments were sequenced and transferred via the Gateway system sequentially into pDONR and pKGWFS7 vectors (Invitrogen). The resulting plasmids were used to transform Agrobacterium and Arabidopsis. Homozygous lines were selected for each isoform.

### Staining and Imaging of GUS

GUS staining was performed according to Lagarde et al. (1996). Images were captured using a Nikon Eclipse 800 microscope.

### Crossing of Apk Mutants

Single mutants apk1-1 and apk2-1, apk1-1 and apk3-1, apk1-1 and apk4-1, apk2-1 and apk3-1, apk2-1 and apk4-1, and apk3-1 and apk4-1 were crossed to generate double mutants. Segregating F2 seeds were screened by PCR at one locus, plants homozygous for the insertion at this locus were then screened at the second loci. Doubly homozygous individuals were obtained for all six possible mutant combinations.

### Glucosinolate Analysis of Apk Mutants

Glucosinolates were extracted from 50 mg crushed freeze-dried leaf material or from 20 mg of seed. Quantification of intact glucosinolates followed the protocol described in Burow et al., (2006). Briefly, samples were extracted in 80% methanol (v:v) containing the internal standard sinigrin. After centrifugation, the supernatants were loaded onto columns of DEAE-Sephadex. The columns were washed with 80% methanol (v:v) and water. Bound intact glucosinolates were desulphated with sulphatase overnight. Desulphoglucosinolate extracts after elution of the columns were separated by reversed-phase HPLC and quantified by UV absorption at 229 nm relative to the internal standard using response factors.

Native desulphoglucosinolates in the plant tissues were extracted as described for intact glucosinolates above. However, desulphoglucosinolates do not bind to DEAE-Sephadex. The flow through from loading the extract onto DEAE-Sephadex columns and the following washing step with 80% methanol (v:v) were collected and combined and analysed by HPLC-UV as described above. Desulphoglucosinolates were quantified relative to desulphosinigrin standard which was added to the extract at an equal concentration to sinigrin.

Identity of intact glucosinolates and of desulphoglucosinolates in the plant extracts were confirmed by liquid chromatography-mass spectrometry on a Bruker Esquire 6000 ion trap mass spectrometer (Bruker Daltonic, Bremen, Germany).

### Jasmonate Analysis of apk Mutants

Jasmonates were quantified from 500 mg fresh crushed leaf tissue. Extraction, HPLC purification and quantification by GC-MS and LC-ESI-SRM, respectively, were performed as described by Miersch et al. (2008).

### Isolation, Purification and Quantification of IAA

Fresh plant material (about 500 mg) was homogenized in 10 ml methanol with appropriate amounts of (¹³C₆)IAA (100 ng, Cambridge Isotope Laboratories) as internal standard. The homogenate was filtered and placed on a cartridge filled with DEAE-Sephadex A-25 (3 ml gel, acetate form, methanol). The column was washed with 3 ml methanol, with 3 ml of 0.1 M HOAc in MeOH and with 3 ml of 1 M acetic acid in methanol. Fractions eluted with 3 ml of 1.5 M and 9 ml of 3 M acetic acid in methanol were combined, evaporated and separated by RP-18 HPLC.

The HPLC analyses were performed using an LC 1100 series Agilent system equipped with a Lichrospher 100 RP-18 column (250×4 mm, 5 μm, Merck). The chromatographic conditions were as follows: flow rate at 1 ml min⁻¹; detection at 210 nm; solvent A: 0.2% (v/v) acetic acid in water, solvent B: methanol. The gradient was from 40% B to 100% B in 25 min. Fraction corresponding to authentic IAA (10-11.5 min) was collected, concentrated in vacuo, methylated with ethereal diazomethane and stored at −20° C. prior to the GC-MS analysis.

The quantitative IAA analyses were carried out on a Polaris Q instrument (Thermo-Finnigan) using the following parameters: 100 eV, positive chemical ionisation (PCI), 1.5 ml methane as ionisation gas, ion source temperature 200° C., column: Rtx-MS (Restek, Germany) 15 m×0.25 mm, 0.25 μm film thickness, crossbond 5% diphenyl−95% dimethyl polysiloxane, injection temperature: 220° C/, interface temperature: 250° C.; helium: 1 mL min⁻¹; splitless injection; Column temperature program: 1 min 60° C., 20° C. min⁻¹ to 300° C., 5 min 300° C. The retention times of methyl esters were obtained at 7.92 min for the internal standard (¹³C₆)IAA and of IAA. Fragments at m/z 136 (¹³C₆)IAA-Me and at m/z 130 (IAA-Me) were used for quantification.

### Determination of Other Metabolites

Hydrophilic metabolites were extracted from leaves of Arabidopsis plants according to Wirtz and Hell (2003). Thiols and amino acids, including O-acetylserine, were quantified after derivatization with monobromobimane (Calbiochem, EMD Chemicals) and AccQ-Tag reagent (Waters), respectively. Anions were separated and quantified after 10-fold dilution in water according to Wirtz and Hell (2007). APS was derivatized with chloroacetaldehyde and separated by HPLC as described in Bürstenbinder et al. (2007) for adenosine compounds using the same HPLC-system. The separation method for APS was developed according to Haink and Deussen (2003).

### Example 1

The Arabidopsis thaliana Genome Encodes Four Functional APS Kinases

In Arabidopsis, two APS kinase enzymes were previously shown to form PAPS from APS and ATP (Lee & Leustek, 1998; Lillig et al., 2001). In addition to these two isoforms, Apk1 (At2g14750) and Apk2 (At4g39940), the completed Arabidopsis genome sequence contains two further predicted isoforms, annotated Apk3 (At3g03900) and Apk4 (At5g67520) (Table 1). The Apk isoforms are 62-72% identical on the amino acid level, with approx. 46% identity to Apk from Escherichia coli (Table S1). Web-based subcellular prediction programs (ChloroP and TargetP) (Emanuelsson et al., 2000; Nielsen et al., 1997) predict that Apk3 is cytosolic, and that Apk1, -2 and -4 possess putative N-terminal plastidic target peptides (Table 1). To determine whether these additional genes encode functional Apk isoforms, the cDNAs for each isoform (minus their putative target peptides) were expressed in E. coli using the pET expression vector and the recombinant proteins purified using the His-Tag system. The previously established Apk activity assay of Renosto et al., (1984) was used to test the activity of the four recombinant enzymes. All four of the proteins exhibited activity, converting APS to PAPS in the in vitro assay with similar kinetic properties (Table 1). Thus, Arabidopsis contains four functional APS kinase isoforms encoded by SEQ ID NO: 1-4.

### Example 2

### Apk Isoforms Show Different Tissue Specific Expression

To examine the tissue-specific expression of the four isoforms, APKₚᵣₒ:APK:GUS fusion constructs were used to stably transform Arabidopsis. GUS staining was clearly localised to the veins of the leaves of transgenic plants for all four isoforms (FIG. 3A). In roots, APK1, -2 and -3 are expressed in and around the vasculature, APK4 expression can be seen but is weak (FIG. 3B). In the root tip, APK2 expression appears to be restricted to the quiescent centre and APK4 to the vasculature. APK1 and 3 show more general expression in most cell types of the root tip (FIG. 3C). APK2 is absent from pollen, but APK1, -3 and -4 are strongly expressed in pollen grains (FIG. 3D, E). In developing seeds, APK1 and 2 appear to be strongly and specifically expressed in the funiculus (FIG. 3F). Clearly, the individual isoforms show very different expression patterns indicating only partial redundancy in function.

### Example 3

### Single Knockouts of Apk Do Not Show Visible Phenotypic Changes

To investigate whether the individual Arabidopsis Apk isoforms have specific functions we utilised available reverse genetics resources. Two independent SALK T-DNA insertion alleles for each isoform in the Col-0 genetic background were obtained from NASC (FIG. 4A). Homozygous lines were isolated by PCR, and the positions of the insertions were confirmed. The loss of transcripts in the T-DNA lines was established by RT-PCR (FIG. 4B). These null T-DNA insertion lines did not show any obvious phenotypic differences compared to Col-0. To determine whether disruption of the genes for individual Apk isoforms has any effect on sulphated metabolites we determined the total glucosinolate levels in rosette leaves of the 5-week-old mutants grown at short days (FIG. 4C). There were no significant effects on the levels of total sulphated glucosinolates in any of the single knock-out lines. The results thus revealed that none of the four Apk isoforms on its own is essential for provision of sufficient PAPS for glucosinolate synthesis.

### Example 4

### Double Knockout of Apk1 and Apk2 has a Semi-Dwarf Phenotype and Contains Reduced Levels of Glucosinolates

As there is at least some functional redundancy in the Apk family in Arabidopsis it was necessary to obtain multiple knock-out lines. Crosses were performed between single knockout plants to create all six double mutant combinations. The lines apk1-1, apk2-1, apk3-1 and apk4-1 were used as parents. Segregating F2 individuals were PCR-genotyped at the parental loci; plants homozygous for the T-DNA insertion at both loci were analysed by RT-PCR to confirm lack of the corresponding transcripts (FIG. 5A). All six possible combinations were isolated. Remarkably, the apk1 apk2 mutant displayed a visible semi-dwarf phenotype (FIG. 5B). The mutant plants were smaller than wild type plants (˜70% reduction in fresh weight) over the whole growth period at short (10 h) days (FIG. 5C); at long days they initiated flowering approximately one week later than the wild type. However, flowers appeared normal and seed set and germination were not obviously affected. Analysis of glucosinolates in the rosette leaves of 5-week-old plants of the double insertion lines grown at short days revealed that the total glucosinolate (gls) level in the apk1 apk2 mutant was considerably reduced to 20% of WT levels (FIG. 5D). Gls levels in other double mutant combinations were not affected with the exception of the apk1 apk4 mutant. Surprisingly this double mutant contained 30% more total gls (FIG. 5D). The lower gls content in apk1 apk2 plants was observed also in 2-week-old seedlings grown in liquid culture (data not shown). Thus, the decrease of the Apk1/Apk2-catalysed synthesis of activated sulphate strongly affects the rate of vegetative development.

A total of 13 different glucosinolates were identified in the mature leaves: nine aliphatic and four indolic. Individual aliphatic gls were reduced by between 4- and 15-fold in apk1 apk2, with the most abundant 4-methylsulphinylbutyl-gls (4MSOB) being reduced most. Total aliphatic gls were reduced to less than 11% of the level of the WT leaves (FIG. 6A). The indolic-gls were less affected in the rosette leaves of the apk1 apk2 plants, the total indolic-gls content was reduced to 30% of those of control plants. Levels of individual modified indolic gls were reduced 3 to 7-fold in the leaves of apk1 apk2 compared to WT leaves.

Gls in the mature seeds of WT and apk1 apk2 plants was also measured (FIG. 6B). A total of 13 gls were identified in the seeds. Total seed gls in apk1 apk2 were present at 7% of the WT level. Individually, the seed gls were reduced between 1.7-fold for I3M to 35-fold for several aliphatic ones. 4MOI3M and 4MOI3M were undetectable in seeds of apk1 apk2 at all.

### Example 5

### Desulpho-Precursors Accumulate in Apk1 Apk2 Leaves, but Not in Seeds

Levels of desulphated glucosinolates (ds-gls), the immediate biosynthetic precursors of gls were determined in rosette leaves of 5-week-old apk1 apk2 and WT plants grown at short days and in mature seeds. Ds-gls were present at very low levels in leaves and were absent or below detection limit in seeds of wild-type plants (FIG. 7). However, in the leaves of the apk1 apk2 mutant, ds-gls accumulated to 11-fold higher levels than the sulphated gls in the WT (FIG. 7). This was true for all the ds-gls measured. In the seeds of the mutants, on the other hand, only very low levels of ds-gls were detected (FIG. 7).

### Example 6

Glucosinolate biosynthetic Pathway is Constitutively Upregulated in Apk1 Apk2

The increase in ds-gls levels in the leaves of apk1 apk2 plants suggested that the gls biosynthesis may be upregulated in these plants. We performed a microarray analysis to examine the transcript levels of the glucosinolate biosynthetic pathway and overall alterations of transcriptome in the leaves of apk1 apk2 mutant. The microarray analysis revealed that transcript levels of genes involved in gls synthesis, such as the UGT74B1 (Grubb et al., 2004), CYP83B1 (Bak et al., 2001), CYP79F1 (Reintanz et al., 2001), SOT16, SOT17, and SOT18 (Piotrowski et al., 2004) were significantly increased in the apk1 apk2 plants compared to wild type (Table 2). Most of these results were verified by real time RT-PCR, confirming 2- to 18-fold higher mRNA levels in the apk1 apk2 mutants. Also the genes encoding MYB factors involved in regulation of aliphatic and indolic gls synthesis HAG1/MYB28, HAG3/MYB29, HAG2/MYB76 and HIG1/MYB51, HIG2/MYB122, HIG3/ATR1/MYB34 (Celenza et al., 2005; Gigolashvili et al., 2007a, 2007b; Hirai et al., 2007) were highly induced in the mutant plants. Thus, the reduction in PAPS supply and consequently the reduction in gls levels results in a strong upregulation of the gls biosynthetic pathway.

### Example 7

apk1 apk2 Double Mutant Contains Reduced Levels of Sulphated Jasmonate

To find out whether the limitation of PAPS synthesis in the apk1 apk2 double mutant specifically affects levels of glucosinolates or whether it also has broader effects on other sulphated metabolites, we analysed levels of another sulphated compound, the sulphated 12-hydroxyjasmonate (Gidda et al., 2003). The SOT catalysing its synthesis (AtSOT15) exhibits strict substrate specificity in vitro for the hydroxylated jasmonates 11-OH-JA and 12-OH-JA. Since so far among the sulphated jasmonates only sulphated 12-OH-JA was found at remarkably abundant levels in different plant species (Miersch et al., 2008), we were specifically interested whether reduced levels of PAPS lead to an alteration in the level of sulphated 12-OH-JA (12-HSO₄-JA). Therefore, levels of 12-HSO₄-JA and that of related compounds such as OPDA, JA, 11-OH-JA, 12-OH-JA and 12-O-glucosyl-JA (12-O-Glc-JA) were determined in 5-week-old rosette leaves of the six double mutants and of apk1-1 and apk2-1 single insertion lines which had been crossed to generate the apk1 apk2 double mutant. Similarly to the gls, levels of 12-HSO₄-JA were reduced by 78% in the apk1 apk2 mutant compared to WT (Table 3), and there was a corresponding increase in the levels of the 11-OH-JA and 12-OH-JA and also of 12-O-Glc-JA. Levels of JA itself were not affected, however, its precursor OPDA was decreased by 40%. In contrast to glucosinolates which were affected specifically in the apk1 apk2 mutant, the content of 12-HSO₄-JA was also reduced in other mutants, apk1-1, apk1 apk3, and apk1 apk4, however, only by 20-30% (Table 3). Very surprisingly, in apk2 apk4 and apk3 apk4 the level of 12-HSO₄-JA was higher than in the WT plants. The precursor of 12-HSO₄-JA, 12-OH-JA, accumulated to slightly but significantly higher levels in apk1-1 and most of the double mutants except for apk1 apk3 and apk1 apk4.

### Example 8

Expression Level of PSK Precursors is Elevated in apk1 apk2 Double Mutant

PSK genes encode precursors for the sulphated PSK pentapeptides, which are sulphated at both their tyrosine residues using PAPS as the sulphate donor. We hypothesised that if the levels of the sulphated peptides diminish, the transcript levels of their precursors would be upregulated. Therefore, mRNA level of three PSK precursor genes was examined in the apk1 apk2 double mutant by semiquantitative RT-PCR (FIG. 8) and microarray analysis (data not shown). Levels of PSK2 and PSK4 mRNA were approximately eight- and five-fold increased, respectively, in the apk1 apk2 double mutants, while the other PSK genes were not affected.

### Example 9

apk1 apk2 Double Mutant Accumulates Auxin

Mutations in glucosinolate biosynthesis often result in accumulation of auxin, since the biosynthetic pathways of indolic glucosinolates and IAA have a common intermediate, the indole acetaldoxime (Bak et al., 2001; Grubb & Abel, 2006). To test whether the decrease in glucosinolates in apk1 apk2 mutant affects auxin levels we determined total IAA levels in rosette leaves of 5-week-old plants of all the double knockout lines. Again, only the apk1 apk2 mutant showed a significant difference in IAA content, which was approximately threefold higher than in WT or other mutants (FIG. 8). We compared the root morphology of apk1 apk2 and WT plants grown on vertical agar plates as well as their flower morphology but despite the high IAA levels, the apk1 apk2 plants did not display any typical high-auxin phenotypic alterations.

### Example 10

### Disruption of APS Kinase Strongly Affects Primary Sulphate Assimilation

As APS kinase clearly controls the accumulation of sulphated compounds, we tested whether the selective inactivation of Apk1 and Apk2 affect also the primary sulphate assimilation. Therefore, the levels of thiols were determined in the rosette leaves of 5-week-old plants of all double knockout lines. All genotypes missing APK1 contained significantly higher Cys levels, with the highest one, in apk1 apk2 plants, four-fold increased compared to WT (FIG. 10). The level of glutathione, which is an order of magnitude more abundant than cysteine and γ-glutamylcysteine, was elevated in apk1 apk2 and apk1 apk4 plants. We were also interested whether the reduction in capacity to phosphorylate APS would affect its steady state foliar concentration. Again, the levels of APS were significantly reduced only in the apk1 apk2 plants (FIG. 10C). As the thiols and APS in apk1 apk2 were most affected among the Apk double mutants, we tested in these plants whether the disruption of Apk1 and Apk2 has consequences for other components of sulphur metabolism. Indeed, the apk1 apk2 plants accumulated sulphate and O-acetylserine in the leaves to approx. 3-fold and 4-fold higher levels, respectively than the Col-0 (data not shown).

The microarray analysis confirmed that the primary sulphate assimilation to cysteine is disturbed by the disruption of Apk1 and Apk2. The transcript levels of several sulphate transporters, most notably SULTR2;1, which is responsible for root-to-shoot sulphate transport, was increased in apk1 apk2. Similarly, the mRNA levels of ATPS1 and ATPS3 isoforms of ATP sulphurylase, APR1, and sulphite reductase were significantly higher (Table S2). Indeed, the ATP sulphurylase enzyme activity was approximately two-fold increased in leaves of apk1 apk2 plants compared to Col-0, however, no changes were seen in APS reductase activity. Genes encoding components of cysteine synthase complex, serine acetyltransferase and O-acetylserine(thiol)lyase were not dramatically affected, whereas the transcript levels of two genes encoding enzymes of glutathione biosynthesis were slightly but significantly elevated.

To identify which other metabolic pathways are affected by the reduction in PAPS synthesis we determined levels of various metabolites not containing sulphur and explored the microarrays. Although cysteine levels were highly increased in apk1 apk2 plants, the levels of other amino acids were not substantially affected. The misbalance in ionic composition was confirmed as phosphate but not nitrate significantly accumulated in the apk1 apk2 plants. Another metabolites significantly different in apk1 apk2 plants compared to wild-type plants were ATP and ADP. The microarray analysis revealed that despite the semi-dwarf phenotype of the apk1 apk2 plants, only 176 genes were found to be differentially expressed (fold-change threshold 2-fold, q value threshold 0.05). Out of these, 129 genes were more highly expressed in the leaves of the apk1 apk2 while 47 more highly expressed in the WT leaves. Of these, 15 genes are part of the glucosinolate network (Table 2) and six are involved in sulphur metabolism, leaving 155 affected genes from other processes. Iterative group analysis using the Aracyc classification of metabolic pathways confirmed that genes of the glucosinolate biosynthesis pathway are over-represented in the up-regulated group of genes and revealed that the same is true for genes involved in IAA biosynthesis, leucine biosynthesis, NAD biosynthesis, cytokinin biosynthesis, glucoside biosynthesis and jasmonic acid biosynthesis (Data not shown). The same analysis also revealed several metabolic pathways which are down-regulated in the mutant, namely pathways of cellulose biosynthesis, cytokinin glucoside biosynthesis and glycine biosynthesis and degradation.

TABLE 7

Primer sequences for creation of transgenic plants
expressing Apk promoter-coding sequence-GFP

| Promoter/ | | |
|---|---|---|
| gene | 5′ Primer^{a} | 3′ Primer |
| Apk1 | ACTAGTTTCAGTAGCAATCTAAGTATGG | GAATTCTGCTTGAAGATAACCCTTGTTATC |
| | SEQ ID NO:25 | SEQ ID NO:26 |
| Apk2 | CTCGAGTTTGGAGGAGGAGACTAACAAC | TCTAGAGCCCTCAAGATAACCTTTGTTTTG |
| | SEQ ID NO:27 | SEQ ID NO:28 |
| Apk3 | ACTAGTAAATCTGGTTTACTTCATGC | GGATCCCTCGTTTTGAAGGAAACCTTTG |
| | SEQ ID NO:29 | SEQ ID NO:30 |
| Apk4 | GTCGACCCTTACAGGTTTTGACAACAGTC | GTCGACCATACAATTACGTGATTTTGTGG |
| | SEQ ID NO:31 | SEQ ID NO:32 |

^{a}The underlined sequences correspond to the restriction sites used for fusion construction.

### Example 11

### Origin of Seed Glucosinolate

The finding that ds-gls do not accumulate in the seeds of apk1 apk2 supports the hypothesis that seed glucosinolates are imported into the seeds and not synthesised there. The origin of the seed gls is, however, unclear with siliques and leaves proposed as the source organs (Magrath (1993), Du (1998)). In apk1 apk2 plants gls synthesis is reduced due to a reduction in the availability of PAPS. PAPS production is restored in apk1 apk2 in an organ-specific manner by expressing Apk1 under different promoters as described in Table 8. The transgenic plants are analysed for the accumulation of gls and ds-gls in leaves and seeds. Organ-specific expression of Apk1 is confirmed by RT-PCR. These experiments determine if seeds are capable of gls synthesis and which tissues serve as the gls source for the transport into seeds.

TABLE 8
Promoters for restoration of APK activity in apk1 apk2
plants and their tissue specificity.

| | Organ/tissue | | |
|---|---|---|---|
| Promoter | specificity | reference | notes |
| APK1 | root and leaf | Mugford et al., | control, |
| | vasculature, leaf | (2009) | complementation of |
| | epidermis, pollen, | | phenotype |
| | funiculus | | |
| LeB4 | seeds, both embryo | Baumlein et al. | verify that seeds do not |
| | and endosperm | (1991) | synthesise gls |
| SULTR1; 2 | roots | Yoshimoto et al. | |
| | | (2002) | |
| STLS1 | leaves | Stockhaus et al. | |
| | | (1989 | |
| AAP2 | leaf and silique | Kwart et al. (1993) | |
| | vasculature | | |
| SHATTERPROOF1 | silique valves | Liljegren et al. | |
| | | (2000) | |
| SEEDSTICK | funiculus | Pinyopich et al. | |
| | | (2003) | |

### Example 12

### Organ-Specific Manipulation of gls Content

### Reduction of Seed gls Levels

As previously discussed glucosinolates are an important part of plant pathogen defence and therefore their high concentration in vegetative tissues is a highly desirable trait. In seeds, high gls levels reduce the quality of rapeseed press cakes for animal feeding. A reduction of Apk expression leads to decrease in the levels of all gls, without the high auxin phenotypes usually associated with alterations of (mostly indolic) gls (Chen (2201), Grubb (2004)). The total loss of Apk1 and Apk2 was associated with a semi dwarf phenotype.

Therefore, to specifically reduce seed gls the expression of apk1 and apk2 in the tissue producing seed gls is reduced using an artificial miRNA [www.weigelworld.org] under the control of a tissue-specific promoter. An amiRNA which targets both Apk1 and Apk2 is used to achieve a similar reduction in seed gls to that in the Apk1 Apk2 plants. To verify correct functioning of the amiRNA, it is expressed under the constitutive 35S promoter in WT Arabidopsis and compared to gls levels in leaves and seeds of the transgenic plants, WT, and apk1 apk2 mutant. To reduce gls synthesis specifically in the tissue producing gls for transport into seeds the amiRNA is expressed under the control of the corresponding tissue specific promoter. The transgenic plants are analysed for expression of Apk isoforms in different tissues and gls content in leaves, siliques, and seeds. The seed yield and oil content of the seeds is determined as well as levels of amino acids and sugars to verify that no major changes in the seed metabolome are induced. This approach targeting Apk is superior to alternative approaches to the reduction of gls levels, such as the expression a sulphatase under the control of a seed specific promoter (WO/2003/012088). Inactivation of seed gls by desulphation will lead to accumulation of ds-gls in seeds with possible adverse effects on the seed quality. The inventors approach using amiRNA does not target the seeds directly and should not result in any other gross metabolite changes in the seeds apart from a reduction in gls.

### Manipulation of Leaf gls

To increase gls content specifically in the leaves in an effort to increase protection from herbivores, MYB51 and MYB28 can be expressed under the control of the STLS1 promoter. Constitutive expression of these effectors has been shown to result in increased levels of aliphatic-gls and indolic-gls, respectively (Gigolashvili (2007a,b)). Using the STLS1 promoter will restrict the increase in gls to the leaves. This is verified by measuring gls accumulation in different plant organs. Lines with the expected pattern of gls distribution are crossed to obtain STLS1::MYB51/MYB28 double over expressors that will have increased levels of both groups of gls. The gls level is measured in these plants and their growth and development monitored. To stack this trait with the low seed gls trait, STLS1::MYB51/MYB28 is crossed with the amiRNA plants and the gls, growth, and development analysed. This produces Arabidopsis plants with increased gls levels in the leaves to be tested for herbivory resistance.

### Example 13

### Effects of Manipulation of gls Levels on Plant Insect Interactions

The biological function of gls appears to be defence against herbivores, particularly insects (Ratzka (2002). This is increasingly more important nowadays with regard to the serious reduction in availability of pesticides and insecticides resulting from the new EU Thematic Strategy on Pesticides. Indeed, reducing gls levels was shown to induce damage of plants by insect herbivory (Beekwilder (2008)). Therefore, apk1 apk2 were analysed for their interactions with insects (FIG. 11). The experiments were carried out by the JIC entomology facility. The double mutant was assayed to identify whether or not preferential feeding occurred using various test organisms. Specifically the aphid Myzus persicae, the lepidopteran Plutella xylostella and the root-feeding dipteran larvae of Bradysia paupera. Choice chamber assays were undertaken for each of the test species and Arabidopsis lines in 30 replicates. The locations and numbers of test organisms was recorded on a regular basis, as was plant condition using a scale of 1-9 (as per EPPO guideline 135). For the B. paupera assessment, aseptic choice chambers were set up using Petri dishes and agar. Sterile B. paupera eggs were added to the dishes and their feeding locations observed and recorded over the following weeks. As shown in FIG. 11 no significant differences in the numbers of insects found on the WT and mutant plants was seen.

Any developmental differences in the test organisms when fed on the different Arabidopsis lines is to be determined. Thirty individual plants are grown for each of the tested lines and each plant is caged with a single adult M. persicae. A further thirty plants for each line are caged with two 2nd instar P. xylostella larvae each. Over the following days, the condition of the M. persicae and the number of clones is recorded. The condition and size of the P. xylostella larvae is also recorded. For each of the different Arabidopsis lines, four seedlings are planted aseptically into agar within Petri dishes. Thirty replicates are set up for each line. Sterile B. paupera eggs are added to each dish and the growth and development of the larvae observed and recorded over the following weeks.

These experiments allow a detailed dissection of the interplay between different sulphur-containing compounds important in pathogen defence. This is especially important, since the reduction of APK in apk1 apk2 plants resulted in increased thiol levels and induction in transcript levels for genes involved in synthesis of the phytoalexin camalexin, which might represent alternative protection against herbivory. Samples are collected from infested and uninfested plants and analysed for gls content and for levels of alternative sulphur-containing compounds involved in biotic interactions, the phytoalexin camalexin and glutathione. mRNA levels of genes involved in sulphate reduction and genes involved in synthesis of gls, glutathione and camalexin, are also analysed (by RT-PCR) to compare the combined effects of infestation and genetic manipulation on sulphur and gls metabolism.

### Example 14

### Transfer of Knowledge to Brassica Crops

The experiments described herein provide results essential for achieving the manipulation of gls levels in oil-seed rape and other Brassica. As described above, identification of which isoforms of Apk are to be targeted in Brassica to achieve similar reduction in gls as in the apk1 apk2 plants is the first step. Relevant data for one of the B. napus genomic components, the A genome, is to be obtained by analysis on B. rapa, which itself is an important vegetable crop. Analysis of the available EST and genomic sequences has indicated a presence of 6 Apk isoforms in B. rapa. Since in Arabidopsis Apk1 and Apk2 were expressed at significantly higher levels than Apk3 and Apk4, comparisons of the expression of the 6 Apk isoforms in leaves, stems, and siliques of B. rapa by qRT-PCR is undertaken. Three isoforms with the highest transcript levels are to be selected as targets for the down regulation using the amiRNA approach. The amiRNA is expressed in B. rapa under the control of 35S promoter. The transgenic plants (at least 5 independent lines) are analysed for expression of the APK family and gls content in leaves and seeds. A significant reduction in gls in all plant parts correlating with reduction in APK expression levels is expected. Such plants represent an excellent basis for further genetic manipulations to restore the gls levels in vegetative tissues but not the seeds

### REFERENCES

Amano, Y., Tsubouchi, H., Shinohara, H., Ogawa, M., and Matsubayashi, Y. (2007). Tyrosine-sulphated glycopeptide involved in cellular proliferation and expansion in Arabidopsis. Proc. Natl. Acad. Sci. USA. 104, 18333-18338.
Andréasson, E., Bolt Jorgensen, L., Höglund, A. S., Rask, L., and Meijer, J. (2001). Different myrosinase and idioblast distribution in Arabidopsis and Brassica napus. Plant Physiol. 127, 1750-1763.
Bak, S., Tax, F. E., Feldmann, K. A., Galbraith, D. W., and Feyereisen, R. (2001). CYP83B1, a cytochrome P450 at the metabolic branch point in auxin and indole glucosinolate biosynthesis in Arabidopsis. Plant Cell 13, 101-111.
Barlier, I., Kowalczyk, M., Marchant, A., Ljung, K., Bhalerao, R., Bennett, M., Sandberg, G., and Bellini, C. (2000). The SUR2 gene of Arabidopsis thaliana encodes the cytochrome P450 CYP83B1, a modulator of auxin homeostasis. Proc. Natl. Acad. Sci. USA 97, 14819-14824.
Barth, C., and Jander, G. (2006). Arabidopsis myrosinases TGG1 and TGG2 have redundant function in glucosinolate breakdown and insect defense. Plant J. 46, 549-562.
Bäumlein et al. (1991). Mol Gen Genet 225, 121-128.
Bednarek, P., Pilewska-Bednarek M., Svato A., Schneider B., Doubsk J., Mansurova M., Humphry M, Consonni C., Panstruga R., Sanchez-Vallet A., Molina A., and Schulze-Lefert P. (2009). A glucosinolate metabolism pathway in living plant cells mediates broad-spectrum antifungal defense. Science 323, 101-106.
Beekwilder, J., van Leeuwen, W., van Dam, N. M., Bertossi, M., Grandi, V., Mizzi, L., Soloviev, M., Szabados, L., Molthoff, J. W., Schipper, B., Verbocht, H., de Vos, R. C., Morandini, P., Aarts, M. G., and Bovy, A. (2008). The impact of the absence of aliphatic glucosinolates on insect herbivory in Arabidopsis. PLoS ONE 3, e2068.
Breitling, R., Amtmann, A., and Herzyk, P. (2004). Iterative Group Analysis (iGA): A simple tool to enhance sensitivity and facilitate interpretation of microarray experiments. BMC Bioinformatics 5, 34.
Brown, P. D., Tokuhisa, J. G., Reichelt, M. and Gershenzon, J. (2003). Variation of glucosinolate accumulation among different organs and developmental stages of Arabidopsis thaliana. Phytochemistry 62, 471-481.
Bürstenbinder, K., Rzewuski, G., Wirtz, M., Hell, R., and Sauter, M. (2007). The role of methionine recycling for ethylene synthesis in Arabidopsis. Plant J. 49, 238-249.
Burow, M., Müller, R., Gershenzon, J. & Wittstock, U. (2006). Altered glucosinolate hydrolysis in genetically engineered Arabidopsis thaliana and its influence on the larval development of Spodoptera littoralis. J. Chem. Ecol. 32, 2333-2349.
Celenza, J. L., Quiel, J. A., Smolen, G. A., Merrikh, H., Silvestro, A. R., Normanly, J., and Bender, J. (2005). The Arabidopsis ATR1 Myb transcription factor controls indolic glucosinolate homeostasis. Plant Physiol. 137, 253-262.
Chen, S., Petersen, B. L., Olsen, C. E., Schulz, A., and Halkier, B. A. (2001). Long-Distance Phloem Transport of Glucosinolates in Arabidopsis. Plant Physiol. 127, 194-201.
Clay N. K., Adio A. M., Denoux C., Jander G., and Ausubel F. M. (2009). Glucosinolate metabolites required for an Arabidopsis innate immune response. Science 323, 95-101.
Clough, S. J., and Bent, A. F. (1998). Floral dip: a simplified method for Agrobacterium-mediated transformation of Arabidopsis thaliana. Plant J. 16, 735-743.
Colbert T, Till B J, Tompa R, Reynolds S, Steine M N, Yeung A T, McCallum C M, Comai L, Henikoff S. (2001) High-throughput screening for induced point mutations. Plant Physiol. June; 126(2):480-4.
Cumming, M., Leung, S., McCallum, J., and McManus, M. T. (2007). Complex formation between recombinant ATP sulfurylase and APS reductase of Allium cepa (L.). FEBS Lett. 581, 4139-4147.
Delarue, M., Prinsen, E., Onckelen, H. V., Caboche, M., and Bellini, C. (1998). Sur2 mutations of Arabidopsis thaliana define a new locus involved in the control of auxin homeostasis. Plant J. 14, 603-611.
Du, L., and Halkier, B. A. (1998). Biosynthesis of glucosinolates in the developing silique walls. Phytochemistry 48, 1145-1150.
Emanuelsson, O., Nielsen, H., Brunak, S. and von Heijne, G. (2000). Predicting subcellular localization of proteins based on their N-terminal amino acid sequence. J. Mol. Biol. 300, 1005-1016.
Fahey, J. W., Zalcmann, A. T., and Talalay, P. (2001). The chemical diversity and distribution of glucosinolates and isothiocyanates among plants. Phytochemistry 56, 55-51.
Falk, K. L., Tokuhisa, J. G., and Gershenzon, J. (2007). The effect of sulfur nutrition on plant glucosinolate content: Physiology and molecular mechanisms. Plant Biology 5, 573-581.
Fire A, Xu S, Montgomery M, Kostas S, Driver S, Mello C (1998). Potent and specific genetic interference by double-stranded RNA in Caenorhabditis elegans. Nature 391: 806-11.
Gidda, S. K., Miersch, O., Levitin, A., Schmidt, J., Wasternack, C., and Varin, L. (2003). Biochemical and molecular characterization of a hydroxyjasmonate sulfotransferase from Arabidopsis thaliana. J. Biol. Chem. 278, 17895-17900.
Gigolashvili, T., Berger, B., Mock, H. P., Müller, C., Weisshaar, B., and Flügge, U. I. (2007a). The transcription factor HIG1/MYB51 regulates indolic glucosinolate biosynthesis in Arabidopsis thaliana. Plant J. 50, 886-901.
Gigolashvili, T., Yatusevich, R., Berger, B., Müller, C., and Flügge, U. I. (2007b). The R2R3-MYB transcription factor HAG1/MYB28 is a regulator of methionine-derived glucosinolate biosynthesis in Arabidopsis thaliana. Plant J. 51, 247-261.
Gigolashvili, T., Engqvist, M., Yatusevich, R., Müller, C., and Flügge, U. I. (2008). HAG2/MYB76 and HAG3/MYB29 exert a specific and coordinated control on the regulation of aliphatic glucosinolate biosynthesis in Arabidopsis thaliana. New Phytol. 177, 627-642.
Goda, E., Kamiyama, S., Uno, T., Yoshida, H., Ueyama, M., Kinoshita-Toyoda, A., Toyoda, H., Ueda, R., and Nishihara, S. (2006). Identification and characterization of a novel Drosophila 3′-phosphoadenosine 5′-phosphosulfate transporter. J Biol. Chem. 281, 28508-28517.
Grubb, C. D., Zipp, B. J., Ludwig-Müller, J., Masuno, M. N., Molinski, T. F., and Abel, S. (2004). Arabidopsis glucosyltransferase UGT74B1 functions in glucosinolate biosynthesis and auxin homeostasis. Plant J. 40, 893-908.
Grubb, C. D., and Abel, S., (2006). Glucosinolate metabolism and its control. Trends Plant Sci. 11, 89-100.
Haink, G., and Deussen, A. (2003). Liquid chromatography method for the analysis of adenosine compounds. J. Chromatogr. B Analyt. Technol. Biomed. Life Sci. 784, 189-193.
Halkier, B. A., and Gershenzon, J. (2006). Biology and biochemistry of glucosinolates. Annu. Rev. Plant Biol. 57, 303-333.
Hasan, M., Friedt, W., Pons-Kühnemann, J., Freitag, N. M., Link, K., and Snowdon, R. J. (2008) Association of gene-linked SSR markers to seed glucosinolate content in oilseed rape (Brassica napus ssp. napus). Theor. Appl. Genet. 116, 1035-1049.
Hasan, M., Seyis, F., Badani, A. G., Pons-Kuhnemann, J., Lühs, W., Friedt, W., and Snowdon, R. J. (2006). Analysis of genetic diversity in the Brassica napus L. gene pool using SSR markers. Genet. Resour. Crop Evol. 53, 793-802.
Hirai, M. Y., Fujiwara, T., Awazuhara, M., Kimura, T., Noji, M., and Saito, K. (2003). Global expression profiling of sulfur-starved Arabidopsis by DNA macroarray reveals the role of O-acetyl-1-serine as a general regulator of gene expression in response to sulfur nutrition. Plant J. 33, 651-663.
Hirai, M. Y., Sugiyama, K., Sawada, Y., Tohge, T., Obayashi, T., Suzuki, A., Araki, R., Sakurai, N., Suzuki, H, Aoki, K., Goda, H., Nishizawa, O. I., Shibata, D., and Saito, K. (2007). Omics-based identification of Arabidopsis Myb transcription factors regulating aliphatic glucosinolate biosynthesis. Proc. Natl. Acad. Sci. USA 104, 6478-6483.
Höfgen, R., and Willmitzer, L. (1988). Storage of competent cells for Agrobacterium transformation. Nucleic Acids Res. 16, 9877.
Kamiyama, S., Suda, T., Ueda, R., Suzuki, M., Okubo, R., Kikuchi, N., Chiba, Y., Goto, S., Toyoda, H., Saigo, K., Watanabe, M., Narimatsu, H., Jigami, Y., and Nishihara, S. (2003). Molecular cloning and identification of 3′-phosphoadenosine 5′-phosphosulfate transporter. J. Biol. Chem. 278, 25958-25963
Kim, J. H., and Jander, G. (2007). Myzus persicae (green peach aphid) feeding on Arabidopsis induces the formation of a deterrent indole glucosinolate. Plant J. 49, 1008-1019.
Klein, M., and Papenbrock, J. (2004). The multi-protein family of Arabidopsis sulphotransferases and their relatives in other plant species. J. Exp. Bot. 55, 1809-1820.
Klein, M., Reichelt, M., Gershenzon, J., and Papenbrock, J. (2006). The three desulfoglucosinolate sulfotransferase proteins in Arabidopsis have different substrate specificities and are differentially expressed. FEBS J. 273, 122-136.
Koncz, C., and Schell, J. (1986). The promoter of T-DNA gene 5 controls the tissue-specific expression of chimaeric genes carried by a novel types of Agrobacterium binary vector. Mol. Gen. Genet. 204, 383-396.
Kopriva, S. (2006). Regulation of sulfate assimilation in Arabidopsis and beyond. Ann. Bot. 97, 479-495.
Kopriva, S., Wiedemann, G., and Reski, R. (2007). Sulfate assimilation in basal land plants—what does genomic sequencing tell us? Plant Biol. 9, 556-564.
Koprivova, A., Suter, M., Op den Camp, R. Brunold, C., and Kopriva, S. (2000). Regulation of sulfate assimilation by nitrogen in Arabidopsis. Plant Physiol. 122, 737-746.
Koroleva, O. A., Davies, A., Deeken, R., Thorpe, M. R., Tomos, A. D., and Hedrich, R. (2000). Identification of a new glucosinolate-rich cell type in Arabidopsis flower stalk. Plant Physiol. 124, 599-608.
Kwart et al. (1993). Plant J 4, 993-1002.
Lagarde, D., Basset, M., Lepetit, M., Conejero, G., Gaymard, F., Astruc, S. and Grignon, C. (1996). Tissue-specific expression of Arabidopsis AKT1 gene is consistent with a role in K⁺ nutrition. Plant J. 9, 195-203.
Lee, S., and Leustek, T. (1998). APS kinase from Arabidopsis thaliana: genomic organization, expression, and kinetic analysis of the recombinant enzyme. Biochem. Biophys. Res. Commun. 247, 171-175.
Liljegren et al. (2000). Nature 404, 766-770.
Lillig, C. H., Schiffmann, S., Berndt, C., Berken, A., Tischka, R., and Schwenn, J. D. (2001). Molecular and catalytic properties of Arabidopsis thaliana adenylyl sulfate (APS)-kinase. Arch. Biochem. Biophys. 392, 303-310.
Lüders, F., Segawa, H., Stein, D., Selva, E. M., Perrimon, N., Turco, S. J., and Häcker, U. (2003). Slalom encodes an adenosine 3′-phosphate 5′-phosphosulfate transporter essential for development in Drosophila. EMBO J. 22, 3635-3644.
Magrath. R., and Mithen, R. (1993). Maternal effects on the expression of individual aliphatic glucosinolates in seeds and seedlings of Brassica napus. Plant Breed. 111, 249-252.
Malitsky, S., Blum, E., Less, H., Venger, I., Elbaz, M., Morin, S., Eshed, Y., and Aharoni, A. (2008). The transcript and metabolite networks affected by the two clades of Arabidopsis glucosinolate biosynthesis regulators. Plant Physiol. 148, 2021-2049.
Mandon, E. C., Milla, M. E., Kempner, E., and Hirschberg, C. B. (1994). Purification of the Golgi adeno sine 3′-phosphate 5′-phosphosulfate transporter, a homodimer within the membrane. Proc Natl Acad Sci USA. 91, 10707-10711.
McCallum C M, Comai L, Greene E A, Henikoff S. (2000) Targeted screening for induced mutations. Nat Biotechnol. 18(4):455-7.
Maruyama-Nakashita, A., Nakamura, Y., Tohge, T., Saito, K., and Takahashi, H. (2006). Arabidopsis SLIM 1 is a central transcriptional regulator of plant sulfur response and metabolism. Plant Cell 18, 3235-3251.
Matsubayashi, Y. and Sakagami, Y. (1996). Phytosulfokine, sulfated peptides that induce the proliferation of single mesophyll cells of Asparagus officinalis L. Proc. Natl. Acad. Sci. USA 93, 7623-7627.
Matsubayashi, Y., Hanai, H., Hara, O. and Sakagami, Y. (1996). Active fragments and analogs of the plant growth factor, phytosulfokine: structure-activity relationships. Biochem. Biophys. Res. Commun. 225, 209-214.
Matsubayashi, Y., Ogawa, M., Kihara, H., Niwa, M. and Sakagami, Y. (2006). Disruption and overexpression of Arabidopsis phytosulfokine receptor gene affects cellular longevity and potential for growth. Plant Physiol. 142, 45-53.
Miersch, O., Neumerkel, J., Dippe, M., Stenzel, I. and Wasternack, C. (2008). Hydroxylated jasmonates are commonly occurring metabolites of jasmonic acid and contribute to a partial switch-off in jasmonate signaling. New Phytol. 177, 114-127.
Mikkelsen, M. D., Naur, P. and Halkier, B. A. (2004). Arabidopsis mutants in the C-S lyase of glucosinolate biosynthesis establish a critical role for indole-3-acetaldoxime in auxin homeostasis. Plant J. 37, 770-777.
Mithen, R., Faulkner, K., Magrath, R., Rose, P., Williamson, G. and Marquez, J. (2003). Development of isothiocyanate-enriched broccoli, and its enhanced ability to induce phase 2 detoxification enzymes in mammalian cells. Theor. Appl. Genet. 106, 727-734.
Mugford S G, Yoshimoto N, Reichelt M, Wirtz M, Hill L, Mugford S T, Nakazato Y, Noji M, Takahashi H, Kramell R, Gigolashvili T, Flügge U I, Wasternack C, Gershenzon J, Hell R, Saito K, Kopriva S. (2009). Disruption of adenosine-5′-phosphosulfate kinase in Arabidopsis reduces levels of sulfated secondary metabolites. Plant Cell. March; 21(3), 910-27.
Nielsen, H., Engelbrecht, J., Brunak, S. and von Heijne, G. (1997). Identification of prokaryotic and eukaryotic signal peptides and prediction of their cleavage sites. Protein Engineering 10, 1-6.
Pedras, M. S., and Montaut, S. (2004). The biosynthesis of crucifer phytoalexins: unprecedented incorporation of a 1-methoxyindolyl precursor. Chem. Commun. (Camb). 21, 452-453.
Piotrowski, M., Schemenewitz, A., Lopukhina, A., Müller, A., Janowitz, T., Weiler, E. W., and Oecking, C. (2004). Desulfoglucosinolate sulfotransferases from Arabidopsis thaliana catalyze the final step in the biosynthesis of the glucosinolate core structure.
J. Biol. Chem. 279, 50717-50725.
Ratzka, A., Vogel, H., Kliebenstein, D. J., Mitchell-Olds, T., and Kroymann, J. (2002). Disarming the mustard oil bomb. Proc. Natl. Acad. Sci. USA. 99, 11223-11228.
Reintanz, B., Lehnen, M., Reichelt, M., Gershenzon, J., Kowalczyk, M., Sandberg, G., Godde, M., Uhl, R., and Palme, K. (2001). Bus, a bushy Arabidopsis CYP79F1 knockout mutant with abolished synthesis of short-chain aliphatic glucosinolates. Plant Cell 13, 351-367.
Renosto, F., Seubert, P. A., and Segel, I. H. (1984). Adenosine 5′-phosphosulfate kinase from Penicillium chrysogenum. Purification and kinetic characterization. J. Biol. Chem. 259, 2113-2123.
Rotte, C., and Leustek, T. (2000). Differential subcellular localization and expression of ATP sulfurylase and 5′-adenylylsulfate reductase during ontogenesis of Arabidopsis leaves indicates that cytosolic and plastid forms of ATP sulfurylase may have specialized functions. Plant Physiol. 124, 715-724.
Rouleau, M., Marsolais, F., Richard, M., Nicolle, L., Voigt, B., Adam, G., and Varin, L. (1999). Inactivation of brassinosteroid biological activity by a salicylate-inducible steroid sulfotransferase from Brassica napus. J. Biol. Chem. 274, 20925-20930.
Sambrook, J., Fritsch, E. F., and Maniatis, T. (1989). Molecular Cloning. A Laboratory Manual, 2nd edn. Cold Spring Harbor Laboratory Press, Cold Spring Harbor, N.Y.
Schilmiller, A. L., and Howe, G. A. (2005). Systemic signaling in the wound response. Curr. Opin. Plant Biol. 8, 36\tilde{9}377
Schöne, F., Rudolph, B., Kirchheim, U., and Knapp, G. (1997). Counteracting the negative effects of oil seed rape and oil seed rape press cake in pig diets. Br. J. Nutr. 78, 947-962.
Schuster, J., Knill, T., Reichelt, M., Gershenzon, J., and Binder, S. (2006). Branched-chain aminotransferase4 is part of the chain elongation pathway in the biosynthesis of methionine-derived glucosinolates in Arabidopsis. Plant Cell 18, 2664-2679.
Sharpe, A. G., and Lydiate, D. J. (2003). Mapping the mosaic of ancestral genotypes in a cultivar of oilseed rape (Brassica napus) selected via pedigree breeding. Genome 46, 461-468.
Shroff, R., Vergara, F., Muck, A., Svatos, A., and Gershenzon, J. (2008). Nonuniform distribution of glucosinolates in Arabidopsis thaliana leaves has important consequences for plant defense. Proc. Natl. Acad. Sci. USA. 105, 6196-6201.
Stenzel, I., Hause, B., Maucher, H., Pitzschke, A., Miersch, O., Ziegler, J., Ryan, C., and Wasternack, C. (2003). Allene oxide cyclase dependence of the wound response and vascular bundle specific generation of jasmonates in tomato—amplification in wound-signaling. Plant J. 33, 57\tilde{7}589
Stockhaus et al. (1989) EMBO J 8, 2445-2451.
Storey, J. D., and Tibshirani, R. (2003). Statistical significance for genome-wide experiments. Proc. Natl. Acad. Sci. USA 100, 9440-9445.
Thangstad, O. P., Gilde, B., Chadchawan, S., Seem, M., Husebye, H., Bradley, D., Bones, A. M. (2004). Cell specific, cross-species expression of myrosinases in Brassica napus, Arabidopsis thaliana and Nicotiana tabacum. Plant Mol. Biol. 54, 597-611.
The Arabidopsis Genome Initiative. (2000). Analysis of the genome sequence of the flowering plant Arabidopsis thaliana. Nature 408, 796-815.
Underhill, E. W., Wetter, L. R., and Chisholm, M. D. (1973). Biosynthesis of glucosinolates. Biochem. Soc. Symp. 38, 303-326.
Valvekens, D., Van Montagu, M., and Van Lijsebettens, M. (1988). Agrobacterium-mediated transformation of Arabidopsis thaliana root explants by using kanamycin selection. Proc. Natl. Acad. Sci. USA 85, 5536-5540.
Wirtz, M., and Hell, R. (2003). Production of cysteine for bacterial and plant biotechnology: Application of cysteine feedback-insensitive isoforms of serine acetyltransferase. Amino Acids 24, 195-203.
Wirtz, M., and Hell, R. (2007). Dominant-negative modification reveals the regulatory function of the multimeric cysteine synthase protein complex in transgenic tobacco. Plant Cell 19, 625-639.
Yan, X., and Chen, S. (2007). Regulation of plant glucosinolate metabolism. Planta 226, 1343-1352.
Yang, H., Matsubayashi, Y., Nakamura, K. & Sakagami, Y. (2001). Diversity of Arabidopsis Genes Encoding Precursors for Phytosulfokine, a Peptide Growth Factor. Plant Physiol. 127, 842-851.
Yoshimoto et al. (2002). Plant J 29, 465-473.

1. A method of producing a plant or a plant cell having an altered level of at least one glucosinolate, said method comprising obtaining a plant or plant cell comprising at least one active Adenosine-5′-Phosphosulphate Kinase (APS kinase, Apk) gene and preventing or inhibiting the production and/or function of the gene product encoded by at least one Adenosine-5′-Phosphosulphate Kinase (APS kinase, Apk) gene.

2. The method according to claim 1, wherein the plant is a member of the family Brassicaceae.

3. The method according to claim 2, wherein said plant is B. carinata—Abyssinian Mustard or Abyssinian Cabbage, B. elongata—Elongated Mustard; B. fruticulosa—Mediterranean Cabbage; B. juncea—Indian Mustard, Brown and leaf mustards, Sarepta Mustard; B. napus—oil seed rape, Canola, Rutabaga (Swede Turnip); Nabicol; B. narinosa—Broadbeaked Mustard; B. nigra—Black Mustard, B. oleracea—Kale; Cabbage, Broccoli, Cauliflower, Kai-lan, Brussels sprouts; B. perviridis—Tender Green, Mustard Spinach; B. rapa (syn B. campestris)—Chinese cabbage, Turnip, Rapini, Komatsuna; B. rupestris—Brown Mustard; B. septiceps—Seventop Turnip; B. tournefortii—Asian Mustard or Arabidopsis.

4. The method according to claim 1, wherein the total level of glucosinolate in the plant or plant cell is reduced.

5. The method according to claim 1, wherein the overall level of glucosinolate in a specific plant tissue or organ is reduced.

6. The method according to claim 4, wherein the level of glucosinolate is reduced by at least 10%, 20%, 30%, 40%, 50%, 60%, 70%, 80%, 90%, or 100%.

7. The method according to claim 5, wherein the level of glucosinolate is reduced by at least 10%, 20%, 30%, 40%, 50%, 60%, 70%, 80%, 90%, or 100%.

8. The method according to claim 1, wherein the plant or plant cell comprises more than one Apk gene.

9. The method according to claim 8, wherein when said plant or plant cell comprises more than one Apk gene, the production of the gene product of at least two Apk genes is prevented or inhibited.

10. The method according to claim 8 which method further comprises determining which Apk genes in said plant or plant cell are complementary and preventing or inhibiting production and/or function of the gene product of said complementary genes.

11. The method according to claim 8, which method further comprises determining which of said Apk genes correspond in function and/or sequence to Arabidopsis Thaliana Apk1 and Apk2 and preventing or inhibiting production and or function of the gene product of said determined genes.

12. The method of claim 11, wherein said Apk genes have at least 50%, 60%, 70%, 75%, 80%, 90%, 95%, 98%, 99% sequence identity with the sequence of Apk1 and/or Apk2 from A. thaliana.

13. The method of claim 12, wherein the polypeptides encoded by said Apk genes have at least 30%, 40%, 50%, 60%, 70%, 75%, 80%, 90%, 95% homology to the polypeptides encoded by Apk1 and/or Apk2 from A. thaliana.

14. The method according to claim 1, wherein the level of at least one glucosinolate is differentially altered in specific tissues or organs of the plant.

15. The method according to claim 1 wherein the overall level of glucosinolate is differentially altered in specific tissues or organs of the plant.

16. The method according to claim 13, wherein the leaves of said plant comprise more glucosinolate than the seeds and/or inflorescences and/or roots of said plant.

17. The method according to claim 14, wherein the leaves of said plant comprise more glucosinolate than the seeds and/or inflorescences and/or roots of said plant.

18. The method according to claim 1, wherein the leaves of said plant maintain a protective effect against pests or infection.

19. The method according to claim 1, wherein the plant produces seed and/or inflorescence and/or root with a reduced level of glucosinolate whilst the leaves maintain a protective effect against pests or infection.

20. The method according to claim 1, wherein preventing or inhibiting the production of the gene product encoded by at least one Adenosine-5′-Phosphosulphate Kinase (APS kinase, Apk) gene is effected by disrupting expression of at least one Adenosine-5′-Phosphosulphate Kinase (APS kinase, Apk) gene.

21. The method according to claim 1, wherein the prevention or inhibition of production and/or function of the Apk gene product occurs post transcriptionally.

22. The method according to claim 20, wherein said prevention or inhibition of production and/or function is effected by iRNA.

23. The method according to claim 1, wherein the prevention or inhibition of production and/or function of the Apk gene product occurs at the transcriptional level.

24. The method according to claim 23, wherein said prevention or inhibition of production and/or function is effected by insertion of at least one nucleotide into an Apk gene or deletion of at least one nucleotide from an Apk gene

25. The method according to claim 23, wherein said prevention or inhibition of production and/or function is effected by introduction of at least one point mutation.

26. A plant or plant cell produced by the method of claim 1.

27. A plant comprising at least one Apk gene product having altered expression.

28. The plant according to claim 27, having a reduced total level of glucosinolate or a reduced overall level of glucosinolate in a specific plant tissue or plant organ.

29. The plant of claim 27, wherein the level of glucosinolate is reduced by at least 10%, 20%, 30%, 40%, 50%, 60%, 70%, 80%, 90%, 100%.

30. The plant according to claim 27 wherein the plant produces seed, inflorescences or roots with a reduced level of glucosinolate whilst the leaves maintain a protective effect against pests or infection.

31. The plant of claim 27, which is a member of the family Brassicaceae.

32. The plant of claim 27, wherein said plant is B. carinata—Abyssinian Mustard or Abyssinian Cabbage; B. elongata—Elongated Mustard; B. fruticulosa—Mediterranean Cabbage; B. juncea—Indian Mustard, Brown and leaf mustards, Sarepta Mustard; B. napus—oil seed rape, Canola, Rutabaga (Swede Turnip); Nabicol; B. narinosa—Broadbeaked Mustard; B. nigra—Black Mustard, B. oleracea—Kale; Cabbage, Broccoli, Cauliflower, Kai-lan, Brussels sprouts; B. perviridis—Tender Green, Mustard Spinach; B. rapa (syn B. campestris)—Chinese cabbage, Turnip, Rapini, Komatsuna; B. rupestris—Brown Mustard; B. septiceps—Seventop Turnip; B. tournefortii—Asian Mustard or Arabidopsis.

33. The plant of claim 27, said plant having a semi dwarf phenotype.

34. A product produced from the plant of claim 26.

35. A seed produced by the plant of claim 26.

36. A product produced from the seed of claim 35.
